SEQUENCE LISTING (87 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 10 aa, Artificial Sequence
  Synthetic peptide
VDPDNEAYEM

SEQ ID NO: 3 - protein, 9 aa, Artificial Sequence
  Synthetic peptide
VDPDNEAYE

SEQ ID NO: 4 - protein, 8 aa, Artificial Sequence
  Synthetic peptide
VDPDNEAY

SEQ ID NO: 5 - protein, 7 aa, Artificial Sequence
  Synthetic peptide
VDPDNEA

SEQ ID NO: 6 - protein, 6 aa, Artificial Sequence
  Synthetic peptide
VDPDNE

SEQ ID NO: 7 - protein, 5 aa, Artificial Sequence
  Synthetic peptide
VDPDN

SEQ ID NO: 8 - protein, 4 aa, Artificial Sequence
  Synthetic peptide
VDPD

SEQ ID NO: 9 - protein, 3 aa, Artificial Sequence
  Synthetic peptide
VDP

SEQ ID NO: 10 - protein, 9 aa, Artificial Sequence
  Synthetic peptide
DPDNEAYEM

SEQ ID NO: 11 - protein, 8 aa, Artificial Sequence
  Synthetic peptide
DPDNEAYE

SEQ ID NO: 12 - protein, 7 aa, Artificial Sequence
  Synthetic peptide
DPDNEAY

SEQ ID NO: 13 - protein, 6 aa, Artificial Sequence
  Synthetic peptide
DPDNEA

SEQ ID NO: 14 - protein, 5 aa, Artificial Sequence
  Synthetic peptide
DPDNE

SEQ ID NO: 15 - protein, 4 aa, Artificial Sequence
  Synthetic peptide
DPDN

SEQ ID NO: 16 - protein, 3 aa, Artificial Sequence
  Synthetic peptide
DPD

SEQ ID NO: 17 - protein, 8 aa, Artificial Sequence
  Synthetic peptide
PDNEAYEM

SEQ ID NO: 18 - protein, 7 aa, Artificial Sequence
  Synthetic peptide
PDNEAYE

SEQ ID NO: 19 - protein, 6 aa, Artificial Sequence
  Synthetic peptide
PDNEAY

SEQ ID NO: 20 - protein, 5 aa, Artificial Sequence
  Synthetic peptide
PDNEA

SEQ ID NO: 21 - protein, 4 aa, Artificial Sequence
  Synthetic peptide
PDNE

SEQ ID NO: 22 - protein, 3 aa, Artificial Sequence
  Synthetic peptide
PDN

SEQ ID NO: 23 - protein, 7 aa, Artificial Sequence
  Synthetic peptide
DNEAYEM

SEQ ID NO: 24 - protein, 6 aa, Artificial Sequence
  Synthetic peptide
DNEAYE

SEQ ID NO: 25 - protein, 5 aa, Artificial Sequence
  Synthetic peptide
DNEAY

SEQ ID NO: 26 - protein, 4 aa, Artificial Sequence
  Synthetic peptide
DNEA

SEQ ID NO: 27 - protein, 3 aa, Artificial Sequence
  Synthetic peptide
DNE

SEQ ID NO: 28 - protein, 6 aa, Artificial Sequence
  Synthetic peptide
NEAYEM

SEQ ID NO: 29 - protein, 5 aa, Artificial Sequence
  Synthetic peptide
NEAYE

SEQ ID NO: 30 - protein, 4 aa, Artificial Sequence
  Synthetic peptide
NEAY

SEQ ID NO: 31 - protein, 3 aa, Artificial Sequence
  Synthetic peptide
NEA

SEQ ID NO: 32 - protein, 5 aa, Artificial Sequence
  Synthetic peptide
EAYEM

SEQ ID NO: 33 - protein, 4 aa, Artificial Sequence
  Synthetic peptide
EAYE

SEQ ID NO: 34 - protein, 3 aa, Artificial Sequence
  Synthetic peptide
EAY

SEQ ID NO: 35 - protein, 4 aa, Artificial Sequence
  Synthetic peptide
AYEM

SEQ ID NO: 36 - protein, 3 aa, Artificial Sequence
  Synthetic peptide
AYE

SEQ ID NO: 37 - protein, 3 aa, Artificial Sequence
  Synthetic peptide
YEM

SEQ ID NO: 38 - protein, 10 aa, Artificial Sequence
  Synthetic peptide
ATGFVKKDQL

SEQ ID NO: 39 - protein, 9 aa, Artificial Sequence
  Synthetic peptide
ATGFVKKDQ

SEQ ID NO: 40 - protein, 8 aa, Artificial Sequence
  Synthetic peptide
ATGFVKKD

SEQ ID NO: 41 - protein, 7 aa, Artificial Sequence
  Synthetic peptide
ATGFVKK

SEQ ID NO: 42 - protein, 6 aa, Artificial Sequence
  Synthetic peptide
ATGFVK

SEQ ID NO: 43 - protein, 5 aa, Artificial Sequence
  Synthetic peptide
ATGFV

SEQ ID NO: 44 - protein, 4 aa, Artificial Sequence
  Synthetic peptide
ATGF

SEQ ID NO: 45 - protein, 3 aa, Artificial Sequence
  Synthetic peptide
ATG

SEQ ID NO: 46 - protein, 9 aa, Artificial Sequence
  Synthetic peptide
TGFVKKDQL

SEQ ID NO: 47 - protein, 8 aa, Artificial Sequence
  Synthetic peptide
TGFVKKDQ

SEQ ID NO: 48 - protein, 7 aa, Artificial Sequence
  Synthetic peptide
TGFVKKD

SEQ ID NO: 49 - protein, 6 aa, Artificial Sequence
  Synthetic peptide
TGFVKK

SEQ ID NO: 50 - protein, 5 aa, Artificial Sequence
  Synthetic peptide
TGFVK

SEQ ID NO: 51 - protein, 4 aa, Artificial Sequence
  Synthetic peptide
TGFV

SEQ ID NO: 52 - protein, 3 aa, Artificial Sequence
  Synthetic peptide
TGF

SEQ ID NO: 53 - protein, 8 aa, Artificial Sequence
  Synthetic peptide
GFVKKDQL

SEQ ID NO: 54 - protein, 7 aa, Artificial Sequence
  Synthetic peptide
GFVKKDQ

SEQ ID NO: 55 - protein, 6 aa, Artificial Sequence
  Synthetic peptide
GFVKKD

SEQ ID NO: 56 - protein, 5 aa, Artificial Sequence
  Synthetic peptide
GFVKK

SEQ ID NO: 57 - protein, 4 aa, Artificial Sequence
  Synthetic peptide
GFVK

SEQ ID NO: 58 - protein, 3 aa, Artificial Sequence
  Synthetic peptide
GFV

SEQ ID NO: 59 - protein, 7 aa, Artificial Sequence
  Synthetic peptide
FVKKDQL

SEQ ID NO: 60 - protein, 6 aa, Artificial Sequence
  Synthetic peptide
FVKKDQ

SEQ ID NO: 61 - protein, 5 aa, Artificial Sequence
  Synthetic peptide
FVKKD

SEQ ID NO: 62 - protein, 4 aa, Artificial Sequence
  Synthetic peptide
FVKK

SEQ ID NO: 63 - protein, 3 aa, Artificial Sequence
  Synthetic peptide
FVK

SEQ ID NO: 64 - protein, 6 aa, Artificial Sequence
  Synthetic peptide
VKKDQL

SEQ ID NO: 65 - protein, 5 aa, Artificial Sequence
  Synthetic peptide
VKKDQ

SEQ ID NO: 66 - protein, 4 aa, Artificial Sequence
  Synthetic peptide
VKKD

SEQ ID NO: 67 - protein, 3 aa, Artificial Sequence
  Synthetic peptide
VKK

SEQ ID NO: 68 - protein, 5 aa, Artificial Sequence
  Synthetic peptide
KKDQL

SEQ ID NO: 69 - protein, 4 aa, Artificial Sequence
  Synthetic peptide
KKDQ

SEQ ID NO: 70 - protein, 3 aa, Artificial Sequence
  Synthetic peptide
KKD

SEQ ID NO: 71 - protein, 4 aa, Artificial Sequence
  Synthetic peptide
KDQL

SEQ ID NO: 72 - protein, 3 aa, Artificial Sequence
  Synthetic peptide
KDQ

SEQ ID NO: 73 - protein, 8 aa, Artificial Sequence
  Synthetic peptide
CPDNEAYE

SEQ ID NO: 74 - protein, 8 aa, Artificial Sequence
  Synthetic peptide
DPDNEAYC

SEQ ID NO: 75 - protein, 8 aa, Artificial Sequence
  Synthetic peptide
CGFVKKDQ

SEQ ID NO: 76 - protein, 10 aa, Artificial Sequence
  Synthetic peptide
PDNEAYEGGC

SEQ ID NO: 77 - protein, 11 aa, Artificial Sequence
  Synthetic peptide
DPDNEAYEGGC

SEQ ID NO: 78 - protein, 19 aa, Artificial Sequence
  Synthetic peptide
PDNEAYERRDPDNEAYGGC

SEQ ID NO: 79 - protein, 19 aa, Artificial Sequence
  Synthetic peptide
DPDNEAYRRPDNEAYEGGC

SEQ ID NO: 80 - protein, 19 aa, Artificial Sequence
  Synthetic peptide
PDNEAYERRTGFVKKDGGC

SEQ ID NO: 81 - protein, 20 aa, Artificial Sequence
  Synthetic peptide
TGFVKKDRRDPDNEAYEGGC

SEQ ID NO: 82 - protein, 4 aa, Artificial Sequence
  Synthetic peptide
RVRR

SEQ ID NO: 83 - protein, 4 aa, Artificial Sequence
  Synthetic peptide
GAGA

SEQ ID NO: 84 - protein, 4 aa, Artificial Sequence
  Synthetic peptide
AGAG

SEQ ID NO: 85 - protein, 4 aa, Artificial Sequence
  Synthetic peptide
KGKG

SEQ ID NO: 86 - protein, 4 aa, Artificial Sequence
  Synthetic peptide
GGGS

SEQ ID NO: 87 - protein, 5 aa, Artificial Sequence
  Synthetic peptide
GGGGS

(19) United States
(12) Patent Application Publication
(10) Pub. No.: US 2023/0355756 A1
(43) Pub. Date: Nov. 9, 2023

(54) ALPHA-SYNUCLEIN VACCINE FOR THE TREATMENT OF SYNUCLEINOPATHIES

(71) Applicant: PROTHENA BIOSCIENCES LIMITED, Dublin 2 (IE)

(72) Inventors: Robin Barbour, Walnut Creek, CA (US); Gene Kinney, Boca Raton, FL (US); Wagner Zago, San Carlos, CA (US)

(21) Appl. No.: 18/245,533

(22) PCT Filed: Aug. 6, 2021
(86) PCT No.: PCT/US2021/045055

§ 371 Date: Mar. 15, 2023

Related U.S. Application Data
(60) Provisional application No. 63/079,819, filed on Sep. 17, 2020.

(51) Int. Cl. A61P 37/04; A61K 39/395

(52) U.S. Cl. CPC A61K 39/3955; A61P 37/04; A61K 2039/6037

(57) ABSTRACT

The disclosure provides peptide compositions and immunotherapy compositions comprising alpha-synuclein peptide. The disclosure also provides methods of treating or effecting prophylaxis of neurodegenerative diseases, such as Parkinson’s disease, dementia with Lewy bodies (DLB), Alzheimer’s disease or other synucleinopathies, with alpha-synuclein deposition in a subject, including methods of clearing deposits, inhibiting or reducing aggregation of alpha-synuclein, blocking the uptake by neurons and inhibiting propagation of alpha-synuclein seeds in a subject having or at risk of developing a neurodegenerative disease containing alpha-synuclein accumulations. The methods include administering to such patients the compositions comprising alpha-synuclein peptide.

### RELATED APPLICATIONS

This application claims the benefit of U.S. Provisional Pat. Application No. 63/079,819, filed Sep. 17, 2020, which is incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the ASCII text file created on May 19, 2021, having the file name “20-1087-WO_Sequence-Listing_ST25.txt” and is 16 kb in size.

### FIELD

The disclosure relates to the technical fields of immunology and medicine, and in particular to the treatment of alpha-synucleinopathies.

### BACKGROUND

Alpha-synucleinopathies, including Parkinson’s disease (PD), dementia with Lewy bodies (DLB), multiple system atrophy (MSA), Alzheimer’s disease (AD) and others are progressive neurological diseases resulting in various kinds of neurodegenerative impairment. Alpha-synuclein, a protein found in neurons and other cells, is a major component of pathology that characterizes these neurodegenerative disorders. The understanding of the normal physiological function of alpha-synuclein is limited, but evidence indicates that soluble forms of the protein may interact with other proteins and certain intracellular membranes. In alpha-synucleinopathies, the alpha-synuclein protein appears to be abnormally aggregated intracellularly, which contributes to disease pathology. There is increasing evidence that certain aggregated forms of alpha-synuclein can be transmitted from neuron to neuron, resulting in a propagation of pathology that causes neuronal dysfunction and loss. Alpha-synuclein (SNCA) misfolding and aggregation can often be accompanied by β-amyloid deposition in some neurodegenerative diseases, including Alzheimer’s disease and Parkinson’s disease.

Accordingly, there exists the need for new therapies and reagents for the prevention or treatment of alpha-synucleinopathies, in particular, therapies and reagents capable of causing an immune response to Alpha-synuclein present in patients.

### SUMMARY

In some embodiments, the disclosure is directed to a peptide or polypeptide including a first peptide comprising 3-10, and in some embodiments 5-10, amino acids from residues 81-140 of SEQ ID NO:01. For example, the peptide may include an amino acid sequence of one of SEQ ID NO:02 to SEQ ID NO:72. In some embodiments, the peptide is from the C-terminal end of alpha-synuclein (residues 111-131 of SEQ ID NO:01) and, as an example, can include any one of SEQ ID NO:02 to SEQ ID NO:37. In some embodiments, the peptide is from the NAC region of alpha-synuclein (residues 83-106 of SEQ ID NO:01) and, as an example, can include any one of SEQ ID NO:38 to SEQ ID NO:72. In some embodiments, the disclosure is directed to a peptide comprising an amino acid sequence selected from the group consisting of any one of SEQ ID NO:73 to SEQ ID NO:81. In some embodiments, the peptide further includes one or more of an N-terminal cysteine and a C-terminal cysteine.

In some embodiments, the peptide may include a linker (for example, to a carrier) at a C-terminal portion of the peptide, or at a N-terminal portion of the peptide, which linker may be an amino acid sequence. A linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the linker is 1 amino acid, 2 amino acids, 3 amino acids, 4 amino acids, 5 amino acids, 6 amino acids, 7 amino acids, 8 amino acids, 9 amino acids, or 10 amino acids. For example, the linker may include an amino acid sequence of GG, GGG, AA, AAA, KK, KKK, SS, SSS, AGAG (SEQ ID NO:84), GG, GAGA (SEQ ID NO:83) and KGKG (SEQ ID NO:85). In addition, the linker to the carrier, if present at the C-terminus, may include a C-terminal cysteine (C), e.g., peptide-XXC, where XX can be GG, AA, KK, SS, GAGA (SEQ ID NO:83), AGAG (SEQ ID NO:84), or KGKG (SEQ ID NO:85). Alternatively, the linker to the carrier, if present at the N-terminus, may include a N-terminal cysteine (C). For example, the sequence may be represented as CXX-peptide, wherein XX and C are independently optional and, if present, XX can be GG, AA, KK, SS, GAGA (SEQ ID NO:83), AGAG (SEQ ID NO:84), or KGKG (SEQ ID NO:85). For example, the polypeptide may include the amino acid sequence of DPDNEAY (SEQ ID NO:12), with an -XXC appended on the C-terminal end and/or a CXX- appended on the N-terminal end, where XX and C are independently optional and, if present, XX can be GG, AA, KK, SS, AGAG, GG, GAGA (SEQ ID NO:83), AGAG (SEQ ID NO:84), and KGKG (SEQ ID NO:85). In some embodiments, the peptide further comprises a blocked amine at the N-terminus.

In other embodiments, the disclosure is directed to an immunotherapy composition including the polypeptides of the disclosure, wherein the polypeptide may be linked to a carrier. The carrier may include serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

Still further, embodiments of the disclosure are directed to a pharmaceutical compositions and formulations comprising the peptides and/or the immunotherapy compositions of the disclosure, and including at least one adjuvant. The adjuvant may be aluminum hydroxide, aluminum phosphate, aluminum sulfate, 3 De-O-acylated monophosphoryl lipid A (MPL) and synthetic analogs thereof, QS-21, QS-18, QS-17, QS-7, TQL1055, Complete Freund’s Adjuvant (CFA), Incomplete Freund’s Adjuvant (IFA), oil in water emulsions (such as squalene or peanut oil), CpG, polyglutamic acid, polylysine, AddaVax™, MF59®, and combinations thereof. In addition, the composition and formulation may include a liposomal formulation, a diluent, and/or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

In addition, the immunotherapy composition may include at least one pharmaceutically acceptable diluent and/or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

The immunotherapy composition may be included in a pharmaceutical composition including the immunotherapy composition and at least one adjuvant such as aluminum hydroxide, aluminum phosphate, aluminum sulfate, 3 De-O-acylated monophosphoryl lipid A (MPL), QS-21, QS-18, QS-17, QS-7, TQL1055, Complete Freund’s Adjuvant (CFA), Incomplete Freund’s Adjuvant (IFA), oil in water emulsions (such as squalene or peanut oil), CpG, polyglutamic acid, polylysine, AddaVax™, MF59®, and combinations thereof.

Embodiments of the disclosure are also directed to nucleic acid sequences encoding the polypeptides and the immunotherapy compositions of the disclosure. The nucleic acids may be included in a nucleic acid immunotherapy composition including the nucleic acid and at least one adjuvant.

Still further, embodiments of the disclosure are directed to a methods for treating or effecting prophylaxis of an alpha-synucleinopathy in a subject, and methods for blocking the uptake of alpha-synuclein by neurons, inhibiting cell-to-cell transmission of alpha-synuclein seeds or inhibiting or reducing aggregation of alpha-synuclein in a subject having or at risk of developing an alpha-synucleinopathy, including Parkinson’s disease (PD), Parkinson’s disease with dementia (PDD), dementia with Lewy bodies (DLB), multiple system atrophy (MSA; including the MSA-parkinsonian (MSA-P) variant and the MSA-cerebellar (MSA-C) variant), Alzheimer’s disease (AD; including the Lewy body variant of AD, and other variants that comprise alpha-synuclein-related pathology), and neurodegeneration with brain iron accumulation (NBIA). The methods include administrating to the subject an immunotherapy composition, a nucleic acids immunotherapy composition, or a pharmaceutical formulation of the disclosure.

The methods of the disclosure may include repeating the administering at least a second time, at least a third time, at least a fourth time, at least a fifth time, or at least a sixth time, and may include repeating the administering at an interval of about bimonthly, of about 21 to about 28 days, of about quarterly, of about biannually, or of about annually.

Still further, methods of the disclosure are directed to inducing an immune response in an animal. The methods include administering to the animal a polypeptide, an immunotherapy composition, a pharmaceutical formulation or a nucleic acid immunotherapy composition of the disclosure in a regimen effective to generate an immune response including antibodies that specifically bind to alpha-synuclein. The immune response may include antibodies that specifically bind to the C-terminal region of alpha-synuclein and/or the NAC region of alpha-synuclein.

In other embodiments, the disclosure is directed to an immunization kit including an immunotherapy composition of the disclosure and may include an adjuvant, wherein the immunotherapy composition may be in a first container and the adjuvant may be a second container.

Still further, the disclosure is directed to a kit including a nucleic acid immunotherapy composition of the disclosure and may include an adjuvant. The nucleic acid may be in a first container and the adjuvant may be in a second container.

### BRIEF DESCRIPTION OF THE FIGURES

FIG. 1 shows the results of an experiment comparing the titers of mouse serum for alpha synuclein single peptide immunogens CPDNEAYE (SEQ ID NO:73), DPDNEAYC (SEQ ID NO:74), and CGFVKKDQ (SEQ ID NO: 75) after four injections. All immunogens comprised a N- or C-terminal cysteine for coupling to maleimide activated CRM197 carrier. QS21 was utilized as an adjuvant in AddaVax squalene-based oil-in-water nano-emulsion.

FIG. 2 shows the results of an experiment measuring the titer of Guinea pig serum for alpha synuclein single peptide immunogen CPDNEAYE (SEQ ID NO:73) and DPDNEAYC (SEQ ID NO:74) after three injections. All immunogens comprised a N- or C-terminal cysteine for coupling to maleimide activated CRM197 carrier. QS21 was used as an adjuvant in AddaVax squalene-based oil-in-water nano-emulsion. Guinea pigs were injected immunogens on day 0, week 3, and week 7, and serum was collected one week after each injection (i.e., week 1, week 4 and week 8). All animals included CRM control samples to ensure a normal immune response.

FIG. 3 shows the results of an experiment comparing the titers of mouse serum for alpha synuclein single peptide immunogens “Single #14” (PDNEAYEGGC (SEQ ID NO:76)), “Single #13” (DPDNEAYEGGC (SEQ ID NO:77)), “Tandem #1” (PDNEAYERRDPDNEAYGGC (SEQ ID NO:78)), “Tandem #2 (DPDNEAYRRPDNEAYEGGC (SEQ ID NO:79)), “Tandem #3” (PDNEAYERRTGFVKKDGGC (SEQ ID NO: 80)), or “Tandem #4” (TGFVKKDRRDPDNEAYEGGC (SEQ ID NO:81). The results show high titers for tandem alpha-synuclein constructs and lower titers for single alpha-synuclein constructs.

FIG. 4 shows the results of an experiment comparing titers of tandem and single alpha-synuclein constructs to monomeric and soluble aggregated alpha-synuclein after the second injection of immunogens of SEQ ID NO:78 (column 1), SEQ ID NO:79 (column 2), SEQ ID NO:80 (column 3), SEQ ID NO:81 (column 4), SEQ ID NO:77 (column 14), SEQ ID NO:76 (column 13).

FIG. 5 shows staining of synuclein pathology in fresh frozen human Parkinson’s brain tissue (A) versus normal tissue (B) using a 1:300 dilution of serum from mice vaccinated with SEQ ID NO:78.

FIG. 6 shows staining of synuclein pathology in fresh frozen human Parkinson’s brain tissue (A) versus normal tissue (B) using a 1:300 dilution of serum from mice vaccinated with SEQ ID NO:79.

FIG. 7 shows staining of synuclein pathology in fresh frozen human Parkinson’s brain tissue (A) versus normal tissue (B) using a 1:300 dilution of serum from mice vaccinated with SEQ ID NO:81.

FIG. 8 shows the ability of antibodies raised against tandem and single alpha-synuclein peptide immunogens in Swiss webster mice to block the binding of alpha-synuclein to a B103 neuronal cell line. “13-x” samples are PDNEAYEGGC (SEQ ID NO:76), “14-x” samples are DPDNEAYEGGC (SEQ ID NO:77), “1-x” samples are PDNEAYERRDPDNEAYGGC (SEQ ID NO: 78), “2-x” samples are DPDNEAYRRPDNEAYEGGC (SEQ ID NO:79), “3-x” samples are PDNEAYERRTGFVKKDGGC (SEQ ID NO:80), and “4-x” samples are TGFVKKDRRDPDNEAYEGGC (SEQ ID NO:81).

FIG. 9 shows the results of an experiment comparing the titers of mouse serum for alpha synuclein single peptide immunogen DPDNEAYEGGC (SEQ ID NO:77), and tandem immunogens DPDNEAYRRPDNEAYEGGC (SEQ ID NO:79) and TGFVKKDRRDPDNEAYEGGC (SEQ ID NO:81). SEQ ID NO:77 was dosed at 2x concentration to control for the being a single alpha-synuclein immunogen compared to the tandem constructs.

FIG. 10 shows staining of synuclein pathology in fresh frozen human Parkinson’s brain tissue using a 1:300 dilution of serum from mice vaccinated with SEQ ID NO:79 (A; “Tandem #2”), SEQ ID NO:81 (B; “Tandem #4”) or SEQ ID NO:77 (C; “2x Single”). Staining appears more prominent for the tandem alpha-synuclein samples.

FIG. 11 shows the ability of antibodies raised against tandem and single alpha-synuclein peptide immunogens in B6 mice to block the binding of alpha-synuclein to a B103 neuronal cell line. “14-x” samples are DPDNEAYEGGC (SEQ ID NO:77), “2-x” samples are DPDNEAYRRPDNEAYEGGC (SEQ ID NO:79), “4-x” samples are TGFVKKDRRDPDNEAYEGGC (SEQ ID NO:81).

### DESCRIPTION

The disclosure provides peptide compositions and immunotherapy compositions comprising an alpha-synuclein peptide. The disclosure also provides methods of treating or effecting prophylaxis of alpha-synucleinopathies, i.e., diseases with alpha-synuclein accumulation or deposition in a subject, including methods of clearing and preventing formation of deposits, inhibiting or reducing aggregation or reducing the abundance of alpha-synuclein, blocking the binding and/or uptake of alpha-synuclein by neurons, inhibiting transmission of alpha-synuclein species between cells, and inhibiting propagation of pathology between brain regions in a subject having or at risk of developing an alpha-synucleinopathy including Parkinson’s disease (PD), Parkinson’s disease with dementia (PDD), dementia with Lewy bodies (DLB), multiple system atrophy (MSA; including the MSA-parkinsonian (MSA-P) variant and the MSA-cerebellar (MSA-C) variant), Alzheimer’s disease (AD); including the Lewy body variant of AD, and other variants that comprise alpha-synuclein-related pathology), and neurodegeneration with brain iron accumulation (NBIA), or other diseases containing alpha-synuclein accumulations. The methods include administering to such patients the compositions comprising an alpha-synuclein peptide.

A number of terms are defined below. As used herein, the singular forms “a,” “an”, and “the” include plural referents unless the context clearly dictates otherwise. For example, the term “a compound” or “at least one compound” can include a plurality of compounds, including mixtures thereof.

Unless otherwise apparent from the context, the term “about” encompasses insubstantial variations, such as values within a standard margin of error of measurement (e.g., SEM) of a stated value. For example, the term “about” as used herein when referring to a measurable value such as a parameter, an amount, a temporal duration, can encompass variations of +/-10% or less, +/-5% or less, or +/-1% or less or less of and from the specified value. Designation of a range of values includes all integers within or defining the range, and all subranges defined by integers within the range. As used herein, statistical significance means p≤0.05.

Compositions or methods “comprising” or “including” one or more recited elements may include other elements not specifically recited. For example, a composition that “comprises” or “includes” a polypeptide sequence may contain the sequence alone or in combination with other sequences or ingredients.

An individual is at increased risk of a disease if the subject has at least one known risk-factor (e.g., age, genetic, biochemical, family history, and situational exposure) placing individuals with that risk factor at a statistically significant greater risk of developing the disease than individuals without the risk factor.

The term “patient” includes human and other mammalian subjects that receive either prophylactic or therapeutic treatment, including treatment naïve subjects. As used herein, the terms “subject” or “patient” refer to any single subject for which treatment is desired, including other mammalian subjects such as, humans, cattle, dogs, guinea pigs, rabbits, and so on. Also intended to be included as a subject are any subjects involved in clinical research trials not showing any clinical sign of disease, or subjects involved in epidemiological studies, or subjects used as controls.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, such as altered gait, as perceived by the subject. A “sign” refers to objective evidence of a disease as observed by a physician.

As used herein, the terms “treat” and “treatment” refer to the alleviation or amelioration of one or more symptoms or effects associated with the disease, prevention, inhibition or delay of the onset of one or more symptoms or effects of the disease, lessening of the severity or frequency of one or more symptoms or effects of the disease, and/or increasing or trending toward desired outcomes as described herein.

The terms “prevention”, “prevent”, or “preventing” as used herein refer to contacting (for example, administering) the peptide(s) or immunotherapy compositions of the present disclosure with a subject before the onset of a disease, with or without alpha-synuclein pathology already present (primary and secondary prevention), thereby delaying the onset of clinical symptoms and/or alleviating symptoms of the disease after the onset of the disease, compared to when the subject is not contacted with the peptide or immunotherapy compositions, and does not refer to completely suppressing the onset of the disease. In some cases, prevention may occur for limited time after administration of the peptide or immunotherapy compositions of the present disclosure. In other cases, prevention may occur for the duration of a treatment regimen comprising administering the peptide or immunotherapy compositions of the present disclosure.

The terms “reduction”, “reduce”, or “reducing” as used herein refer to decreasing the amount of alpha-synuclein present in a subject or in tissue of the subject, or suppressing an increase in the amount of alpha-synuclein present in a subject or in tissue of the subject, which encompasses decreasing or suppressing an increase in (e.g., decreasing the rate of increase) the amount of alpha-synuclein present, accumulated, aggregated, or deposited in the subject or tissue in the subject. In certain embodiments, the decrease in or suppression of an increase in (e.g., decreasing the rate of increase) the amount of alpha-synuclein present, accumulated, aggregated, or deposited in the subject refers to an amount of alpha-synuclein present, accumulated, aggregated, or deposited in the central nervous system (CNS) of the subject. In certain embodiments, the decrease in or suppression of an increase in (e.g., decreasing the rate of increase) the amount of alpha-synuclein present, accumulated, aggregated, or deposited in the subject refers to an amount of alpha-synuclein present, accumulated, aggregated, or deposited in the periphery (e.g., peripheral circulatory system) of the subject. In certain embodiments, the decrease in or suppression of an increase in (e.g., decreasing the rate of increase) the amount of alpha-synuclein present, accumulated, aggregated, or deposited in the subject refers to an amount of alpha-synuclein present, accumulated, aggregated, or deposited in the brain of the subject. In some embodiments, the alpha-synuclein reduced is the pathological form(s) of the alpha-synuclein (e.g. oligomeric or fibrillar alpha-synuclein conglomerates and protofibrillar intermediates of alpha-synuclein oligomers). In yet other embodiments, pathological indicators of neurodegenerative disease and/or synucleinopathies are decreased.

The terms “epitope” or “antigenic determinant” refers to a site on an antigen to which B and/or T cells respond, or to a site on an antigen to which an antibody binds. Epitopes can be formed both from contiguous amino acids or from noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, at least 4, at least 5, at least 6, at least 7, at least 8, at least 9, at least 10, at least 11, at least 12, or at least 13 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996).

An “immunogenic agent” or “immunogen” or “antigen” is capable of inducing an immunological response against itself or modified/processed versions of itself upon administration to an animal, optionally in conjunction with an adjuvant. The terms “immunogenic agent” or “immunogen” or “antigen” refer to a compound or composition comprising a peptide, polypeptide or protein which is “antigenic” or “immunogenic” when administered in an appropriate amount (an “immunogenically effective amount”), i.e., capable of inducing, eliciting, augmenting or boosting a cellular and/or humoral immune response and of being recognized by the products of that response (T cells, antibodies). An immunogen can be a peptide, or a combination of two or more same or different peptides, that includes at least 3, at least 4, at least 5, at least 6, at least 7, at least 8, at least 9, at least 10, at least 11, at least 12, or at least 13 amino acids in a linear or spatial conformation.

A nucleic acid such as DNA or RNA that encodes a peptide immunogen and is used as a vaccine is referred to as a “DNA [or RNA] immunogen,” as the encoded polypeptide is expressed in vivo after administration of the DNA or RNA. The peptide or polypeptide can be recombinantly expressed from a vaccine vector, which can be naked DNA or RNA that comprises the peptide or polypeptide coding sequence operably linked to a promoter, e.g., an expression vector or cassette as described herein.

An immunogen may be effective when given alone or in combination, or linked to, or fused to, another substance (which can be administered at one time or over several intervals). An immunogenic agent or immunogen may include an antigenic peptide or polypeptide that is linked to a carrier as described herein.

A nucleic acid such as DNA or RNA that encodes an antigenic peptide, or polypeptide is referred to as a “DNA [or RNA] immunogen,” as the encoded peptide or polypeptide is expressed in vivo after administration of the DNA or RNA. The peptide or polypeptide can be recombinantly expressed from a vaccine vector, which can be naked DNA or RNA that comprises the peptide or polypeptide coding sequence operably linked to a promoter, e.g., an expression vector or cassette as described herein.

The term “adjuvant” refers to a compound that, when administered in conjunction with an antigen, augments the immune response to the antigen, but when administered alone does not generate an immune response to the antigen. Adjuvants can augment an immune response by several mechanisms including lymphocyte recruitment, stimulation of B and/or T cells, and stimulation of macrophages. An adjuvant may be a natural compound, a modified version of or derivative of a natural compound, or a synthetic compound.

The terms “peptide”, “polypeptide” and “(poly)peptide” are used interchangeably herein and refer to a chain of two or more consecutive amino acids. If and when a distinction is made, context makes the meaning clear. For example, if two or more peptides described herein are joined to make a dimeric or multimeric peptide, polypeptide may be used to indicate “poly” or “more than one” peptide.

The term “pharmaceutically acceptable” means that the carrier, diluent, excipient, adjuvant, or auxiliary is compatible with the other ingredients of a pharmaceutical formulation and not substantially deleterious to the recipient thereof.

The terms “immunotherapy” or “immune response” refer to the development of a beneficial humoral (antibody mediated) and/or a cellular (mediated by antigen-specific T cells or their secretion products) response directed against an alpha-synuclein peptide in a recipient. Such a response can be an active response induced by administration of immunogen (e.g. an alpha-synuclein peptide). A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

### Alpha-synuclein

Alpha-synuclein (SNCA) misfolding and aggregation can often be accompanied by β-amyloid deposition in some neurodegenerative diseases (e.g., Alzheimer’s disease and its subtypes such as the Lewy body variant of AD), and alpha-synuclein exists in several neurodegenerative disorders, including Parkinson’s disease (PD), Parkinson’s disease with dementia (PDD), dementia with Lewy bodies (DLB), multiple system atrophy (MSA; including the MSA-parkinsonian (MSA-P) variant and the MSA-cerebellar (MSA-C) variant), and neurodegeneration with brain iron accumulation (NBIA).

### Peptide Immunogens

An agent used for active immunization can induce in a patient an immune response and can serve as an immunotherapy. Agents used for active immunization can be, for example, the same types of immunogens used for generating monoclonal antibodies in laboratory animals, and may include 3, 4, 5, 6, 7, 8, 9, 10, 11, 12, or 13 or more contiguous amino acids from a region of alpha-synuclein peptide. In each of the embodiments of the peptides described herein, the peptides may comprise, consist, or consist essentially of the recited sequences.

In some embodiments of the disclosure, the immunogen can include an alpha-synuclein peptide comprising 3-10 amino acids from residues 81-140 of the alpha-synuclein polypeptide (SEQ ID NO:01), or from the C-terminal region of alpha-synuclein (residues 111-131 of SEQ ID NO:01), or from the NAC region of alpha-synuclein (residues 83-106 of SEQ ID NO:01). In some embodiments, the peptide fragment is unphosphorylated. In some embodiments, the fragment is phosphorylated at serine (S), threonine (T), and/or tyrosine (Y) phosphorylation sites.

In some embodiments, the alpha-synuclein peptide comprises an amino acid sequence including any one of SEQ ID NO:02 to SEQ ID NO:72. In some embodiments that the immunogen is from the C-terminal region of alpha-synuclein (residues 111-131 of SEQ ID NO:01), the peptides can include any one of SEQ ID NO:02 to SEQ ID NO:37. In some embodiments that the peptide immunogen is from the NAC region of alpha-synuclein (residues 83-106 of SEQ ID NO:01), the peptides can include any one of SEQ ID NO:38 to SEQ ID NO:72. In some embodiments, the peptide comprises an amino acid sequence that includes any one of DPDNEAYE (SEQ ID NO: 11), DPDNEAY (SEQ ID NO: 12), PDNEAYE (SEQ ID NO: 18), ATGFVKK (SEQ ID NO:41), TGFVKKD (SEQ ID NO:48), GFVKKDQ (SEQ ID NO:54), DPDNEAYC (SEQ ID NO:74), CPDNEAYE (SEQ ID NO:73), CGFVKKDQ (SEQ ID NO:75), PDNEAYEGGC (SEQ ID NO:76), DPDNEAYEGGC (SEQ ID NO:77), PDNEAYERRDPDNEAYGGC (SEQ ID NO:78), DPDNEAYRRPDNEAYEGGC (SEQ ID NO:79), PDNEAYERRTGFVKKDGGC (SEQ ID NO:80), or TGFVKKDRRDPDNEAYEGGC (SEQ ID NO:81). Each alpha-synuclein sequence optionally further includes, independently, a C-terminal cysteine and/or an N-terminal cysteine.

In some embodiments, the two or more alpha-synuclein peptides are linked to form an alpha-synuclein polypeptide. The one or more alpha-synuclein peptides can be linked by an intra-peptide linker, which linker is as described above and herein. For example, a polypeptide linker located between the C-terminal of the first peptide and the N terminal of the second peptide. With or without the intra-peptide linker, the alpha-synuclein polypeptide may be arranged in any order. For example, a specific alpha-synuclein peptide (“alpha-synuclein 1”) may be positioned at the N-terminal portion of a dual alpha-synuclein polypeptide and the same or a different alpha-synuclein peptide (for this example, a different alpha-synuclein, “alpha-synuclein 2”) may be positioned at the C-terminal portion of the dual polypeptide. Or, the alpha-synuclein peptides in this example could be arranged in the opposite orientation (alpha-synuclein 2 N-terminal to alpha-synuclein 1). Reference to a first peptide or a second peptide herein is not intended to suggest an order of the alpha-synuclein peptides in embodiments that comprise more than one alpha-synuclein peptide of the immunogens.

In addition, the C-terminal portion and/or the N-terminal portion of the alpha-synuclein peptide or alpha-synuclein polypeptide can include a linker, for example, for conjugating the peptides or the polypeptide to a carrier. Linkers that may couple a peptide or polypeptide to a carrier may include, for example, GG, GGG, KK, KKK, AA, AAA, SS, SSS, GAGA (SEQ ID NO:83), AGAG (SEQ ID NO:84), KGKG (SEQ ID NO:85), and others as described elsewhere herein (e.g., “linker 1” and “linker 2” below), between the peptide or polypeptide and the carrier and may further include a C-terminal cysteine on the C-terminal end of the linker (or directly to the C-terminal end of the peptide or polypeptide sequence where a linker is absent) or a N-terminal cysteine on the N-terminal end of the linker (or directly to the N-terminal end of the peptide or polypeptide sequence where a linker is absent). For example, (poly)peptide-G-G-C, (poly)peptide-K-K-C, (poly)peptide-A-A-C, (poly)peptide-S-S-C, or C-G-G-(poly)peptide, C-K-K-(poly)peptide, C-A-A-(poly)peptide, C-S-S-(poly)peptide. In some embodiments, the immunogen peptides further include a blocked amine at the N-terminus.

When the alpha-synuclein peptides are linked to form an alpha-synuclein polypeptide, the linker may be a cleavable linker. As used herein, the term “cleavable linker” refers to any linker between the antigenic peptides that promotes or otherwise renders the alpha-synuclein polypeptide more susceptible to separation from each other by cleavage (for example, by endopeptidases, proteases, low pH or any other means that may occur within or around the antigen-presenting cell) and, thereby, processing by the antigen-presenting cell, than equivalent peptides lacking such a cleavable linker. In some embodiments, the cleavable linker is a protease-sensitive dipeptide or oligopeptide cleavable linker. In certain embodiments, the cleavable linker is sensitive to cleavage by a protease of the trypsin family of proteases. In some embodiments, the cleavable linker comprises an amino acid sequence including arginine-arginine (Arg-Arg), arginine-valine-arginine-arginine (Arg-Val-Arg-Arg; SEQ ID NO:82), Gly-Ala-Gly-Ala (SEQ ID NO:83), Ala-Gly-Ala-Gly (SEQ ID NO:84), Lys-Gly-Lys-Gly (SEQ ID NO:85), valine-citrulline (Val-Cit), valine-arginine (Val-Arg), valine-lysine (Val-Lys), valine-alanine (Val-Ala), and phenylalanine-lysine (Phe-Lys). In some embodiments, the cleavable linker is arginine-arginine (Arg-Arg).

In some embodiments of the disclosure, the alpha-synuclein polypeptide comprises, consists or consists essentially of an amino acid sequence selected from DPDNEAYE (SEQ ID NO: 11), DPDNEAY (SEQ ID NO: 12), PDNEAYE (SEQ ID NO:18), ATGFVKK (SEQ ID NO:41), TGFVKKD (SEQ ID NO:48), GFVKKDQ (SEQ ID NO:54), CPDNEAYE (SEQ ID NO:73), DPDNEAYC (SEQ ID NO:74) or CGFVKKDQ (SEQ ID NO:75), PDNEAYEGGC (SEQ ID NO:76), DPDNEAYEGGC (SEQ ID NO:77), PDNEAYERRDPDNEAYGGC (SEQ ID NO:78), DPDNEAYRRPDNEAYEGGC (SEQ ID NO:79), PDNEAYERRTGFVKKDGGC (SEQ ID NO:80), or TGFVKKDRRDPDNEAYEGGC (SEQ ID NO:81) wherein XX is optionally appended to the C-terminal end and/or the N-terminal end of SEQ ID NOS: 11, 12, 18, 41, 48, 54, 73, 74, 75, 76, 77, 78, 79, 80, or 81, and a cysteine is optionally appended to the C-terminal end and/or the N-terminal end of SEQ ID NOS: 11, 12, 18, 41, 48, 54, 73, 74, 75, 76, 77, 78, 79, 80, or 81 or if XX is present, to the C-terminal end of a C-terminal XX and/or to the N-terminal end of an N-terminal XX. XX can be GG, AA, KK, SS, GAGA (SEQ ID NO:83), AGAG (SEQ ID NO:84), and KGKG (SEQ ID NO:85).

In some embodiments, the dual alpha-synuclein polypeptide is as follows:
[first peptide]-[linker 1]-[second peptide]-[linker 2]-[Cys],
wherein, the [first peptide] is an alpha-synuclein peptide and the [second peptide] is the same or different alpha-synuclein peptide, and each of [linker 1], [linker 2] and [Cys] is optional, and any [Cys] that is present is a C-terminal [Cys].

In some embodiments, the dual alpha-synuclein polypeptide is as follows:
[Cys]-[linker 2]-[first peptide]-[linker 1]-[second peptide],
wherein, the [first peptide] is an alpha-synuclein peptide and the [second peptide] is the same or different alpha-synuclein peptide, and each of [linker 1], [linker 2] and [Cys] is optional, and any [Cys] that is present is an N-terminal [Cys].

In some embodiments, the dual alpha-synuclein polypeptide is as follows:
[Cys]-[linker 2]-[first peptide]-[linker 1]-[second peptide]-[linker 2]-[Cys],
wherein, the [first peptide] is an alpha-synuclein peptide and the [second peptide] is the same or different alpha-synuclein peptide, and each of [linker 1], [linker 2] and [Cys] is optional, and a [Cys] that is present can be a C-terminal and/or an N-terminal [Cys].

Examples of the alpha-synuclein peptides include any one of SEQ ID NOS:2-81. [Linker 1] is optional, and when present, may be a cleavable linker. A cleavable linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the cleavable linker is 1 amino acid, 2 amino acids, 3 amino acids, 4 amino acids, 5 amino acids, 6 amino acids, 7 amino acids, 8 amino acids, 9 amino acids, or 10 amino acids. In some embodiments, the linker may be a cleavable linker having an amino acid sequence selected from the group consisting of arginine-arginine (Arg-Arg), arginine-valine-arginine-arginine (Arg-Val-Arg-Arg; SEQ ID NO:82), valine-citrulline (Val-Cit), valine-arginine (Val-Arg), valine-lysine (Val-Lys), valine-alanine (Val-Ala), phenylalanine-lysine (Phe-Lys), glycine-alanine-glycine-alanine (Gly-Ala-Gly-Ala; SEQ ID NO:83), alanine-glycine-alanine-glycine (Ala-Gly-Ala-Gly; SEQ ID NO:84), and lysine-glycine-lysine-glycine (Lys-Gly-Lys-Gly; SEQ ID NO:85).

[Linker 2] is optional, and when present is a linker that couples the polypeptide to a carrier. A linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the linker is 1 amino acid, 2 amino acids, 3 amino acids, 4 amino acids, 5 amino acids, 6 amino acids, 7 amino acids, 8 amino acids, 9 amino acids, or 10 amino acids. In some embodiments, the amino acid composition of a linker can mimic the composition of linkers found in natural multidomain proteins, where certain amino acids are overrepresented, underrepresented or equi-represented in natural linkers as compared to their abundance in whole protein. For example, threonine (Thr), serine (Ser), proline (Pro), glycine (Gly), aspartic acid (Asp), lysine (Lys), glutamine (Gln), asparagine (Asn), arginine (Arg), phenylalanine (Phe), glutamic acid (Glu) and alanine (Ala) are overrepresented in natural linkers. In contrast, isoleucine (Ile), tyrosine (Tyr), tryptophan (Trp), and cysteine (Cys) are underrepresented. In general, overrepresented amino acids were polar uncharged or charged residues, which constitute approximately 50% of naturally encoded amino acids, and Pro, Thr, and Gln were the most preferable amino acids for natural linkers. In some embodiments, the amino acid composition of a linker can mimic the composition of linkers commonly found in recombinant proteins, which can generally by classified as flexible or rigid linkers. For example, flexible linkers found in recombinant proteins are generally composed of small, non-polar (e.g. Gly) or polar (e.g. Ser or Thr) amino acids whose small size provides flexibility and allows for mobility of the connecting functional domains. The incorporation of, e.g., Ser or Thr can maintain the stability of the linker in aqueous solutions by forming hydrogen bonds with the water molecules, and therefore can reduce interactions between the linker and the immunogens. In some embodiments, a linker comprises stretches of Gly and Ser residues (“GS” linker). An example of a widely used flexible linker is (Gly-Gly-Ser)n, (Gly-Gly-Gly-Ser)n (SEQ ID NO:86) or (Gly-Gly-Gly-Gly-Ser)n (SEQ ID NO:87), where n=1-3. Adjusting the copy number “n” can optimize a linker to achieve sufficient separation of the functional immunogen domains to, e.g., maximize an immunogenic response. Many other flexible linkers have been designed for recombinant fusion proteins that can be used herein. In some embodiments, linkers can be rich in small or polar amino acids such as Gly and Ser but also contain additional amino acids such as Thr and Ala to maintain flexibility, as well as polar amino acids such as Lys and Glu to improve solubility. See, e.g., Chen, X. et al., “Fusion Protein Linkers: Property, Design and Functionality” Adv Drug Deliv Rev., 15; 65(10): 1357-1369 (203). In certain embodiments, when present, the linker can be an amino acid sequence selected from the group consisting of as GG, GGG, KK, KKK, AA, AAA, SS, SSS, G-A-G-A (SEQ ID NO:83), A-G-A-G (SEQ ID NO:84), and K-G-K-G (SEQ ID NO:85).

[Cys] is optional and can be helpful to conjugate the polypeptide to a carrier. When present, the Cys can be at the C-terminal portion of the polypeptide, or at the N-terminal portion of the polypeptide.

### Peptide-Carrier Immunogens

Alpha-synuclein peptides (and polypeptides thereof) are immunogens in accordance with the disclosure. In some embodiments, the peptides can be linked to a suitable carrier to help elicit an immune response. Accordingly, one or more the peptides and polypeptides of the disclosure can be linked to a carrier. For example, each of the alpha-synuclein (poly)peptides may be linked to the carrier with or without spacer amino acids (e.g., Gly-Gly, Gly-Gly-Gly, Ala-Ala, Ala-Ala-Ala, Lys-Lys, Lys-Lys-Lys, Ser-Ser, Ser-Ser-Ser, Gly-Ala-Gly-Ala (SEQ ID NO:83), Ala-Gly-Ala-Gly (SEQ ID NO:84), or Lys-Gly-Lys-Gly (SEQ ID NO:85)). In certain embodiments, the alpha-synuclein (poly)peptide can be linked to a suitable carrier using a C-terminal cysteine to provide a linker between the (poly)peptide and the carrier. In certain embodiments, the alpha-synuclein (poly)peptide can be linked to a suitable carrier using an N-terminal cysteine to provide a linker between the (poly)peptide and the carrier.

Suitable carriers include, but are not limited to serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking peptide immunogens of the invention to an immunostimulatory polymer molecule (e.g., tripalmitoyl-S-glycerine cysteine (Pam3Cys), mannan (a mannose polymer), or glucan (a β 1-2 polymer)), cytokines (e.g., IL-1, IL-1 alpha and β peptides, IL-2, γ-INF, IL-10, GM-CSF), and chemokines (e.g., MIP1-α and β, and RANTES). Additional carriers include virus-like particles. In some compositions, immunogenic peptides can also be linked to carriers by chemical crosslinking. Techniques for linking an immunogen to a carrier include the formation of disulfide linkages using N-succinimidyl 3-(2-pyridylthio)propionate (SPDP), and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine resides on one protein and an amide linkage through the epsilon-amino on a lysine, or other free amino group in other amino acids. In some embodiments, chemical crosslinking can comprise use of SBAP (succinimidyl 3-(bromoacetamido)propionate), which is a short (6.2 angstrom) cross-linker for amine-to-sulfhydryl conjugation via N-hydroxysuccinimide (NHS) ester and bromoacetyl reactive groups. A variety of such disulfide/amide-forming agents are described by Jansen et al., “Immunotoxins: Hybrid Molecules Combining High Specificity and Potent Cytotoxicity” Immunological Reviews 62:185-216 (February 1982). Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimidomethyl)cy- clohexane-1-carboxylic acid. The carboxyl groups can be activated by combining them with succinimide or 1-hydroxyl-2-nitro-4-sulfonic acid, sodium salt. Virus-like particles (VLPs), also called pseudovirions or virus-derived particles, represent subunit structures composed of multiple copies of a viral capsid and/or envelope protein capable of self-assembly into VLPs of defined spherical symmetry in vivo. (Powilleit, et al., (2007) PLoS ONE 2(5):e415.) Alternatively, peptide immunogens can be linked to at least one artificial T-cell epitope capable of binding a large proportion of MHC Class II molecules, such as the pan DR epitope (“PADRE”). Pan DR-binding peptides (PADRE) are described in US 5,736,142, WO 95/07707, and Alexander, et al, Immunity, 1:751-761 (1994).

Active immunogens can be presented in multimeric form in which multiple copies of an immunogen (peptide or polypeptide) are presented on a carrier as a single covalent molecule. In some embodiments, the carrier includes various forms of the alpha-synuclein peptide. For instance, the alpha-synuclein peptide of the immunogen can include polypeptides that have different alpha-synuclein antigens in different orders, or may be present with or without an intrapeptide linker and/or a linker to a carrier.

In some compositions, the immunogenic peptides can also be expressed as fusion proteins with carriers. In certain compositions, the immunogenic peptides can be linked at the amino terminus, the carboxyl terminus, or internally to the carrier. In some compositions, the carrier is CRM197. In some compositions, the carrier is diphtheria toxoid.

### Nucleic Acids

The disclosure further provides nucleic acids encoding any of the alpha-synuclein peptides as disclosed herein. The nucleic acid immunotherapy compositions as disclosed herein, comprise a nucleic acid sequence encoding one or more alpha-synuclein peptides as disclosed herein. For example, the alpha-synuclein peptide includes a sequence that is 3-10 amino acid residues in length and from residues 81-140 of SEQ ID NO:01. Accordingly, a nucleic acid encoding any of SEQ ID NOS:2-81 provides an immunogen and a component of a pharmaceutical composition of the disclosure. Likewise, one or more nucleic acids encoding any of the alpha-synuclein sequences may include the codons for an N-terminal or C-terminal dipeptide. The peptide sequences may be encoded by the same or separate nucleic acid sequences that may also encode a linker to the carrier and an N- or C-terminal cysteine as described herein. In addition, when a single nucleic acid sequence encodes more than one alpha-synuclein peptide, the sequence may also encode an intra-peptide linker or cleavable linker as described herein.

The nucleic acid compositions described herein (pharmaceutical compositions) can be used in methods for treating or effecting prophylaxis and/or prevention of alpha-synucleinopathies. In certain embodiments, the nucleic acid immunotherapy compositions as disclosed herein provide compositions for treating or effecting prophylaxis of alpha-synucleinopathies in a subject. In some embodiments, the nucleic acid immunotherapy compositions as disclosed herein provide compositions for blocking the uptake of alpha-synuclein by neurons, inhibiting cell-to-cell transmission of alpha-synuclein seeds and/or inhibiting or reducing aggregation of alpha-synuclein in a subject. In some embodiments, the nucleic acid immunotherapy compositions provide for reducing alpha-synuclein in a subject and/or in the tissue of the subject. In another embodiment, the nucleic acid immunotherapy compositions as disclosed herein provide compositions for reducing alpha-synuclein in the brain of a subject. In some embodiments, the alpha-synuclein reduced by the immunotherapy compositions is the pathological form(s) of the alpha-synuclein (e.g. oligomeric or fibrillar alpha-synuclein conglomerates and protofibrillar intermediates of alpha-synuclein oligomers). In yet other embodiment, pathological indicators of neurodegenerative disease and/or β-amyloidopathies are decreased by the immunotherapy compositions.

A nucleic acid such as DNA that encodes an immunogen and is used as a vaccine can be referred to as a “DNA immunogen” or “DNA vaccine” as the encoded polypeptides are expressed in vivo after administration of the DNA. DNA vaccines are intended to induce antibodies against the proteins of interest they encode in a subject by: integrating DNA encoding the proteins of interest into a vector (a plasmid or virus); administering the vector to the subject; and expressing the proteins of interest in the subject in which the vector has been administered to stimulate the immune system of the subject. A DNA vaccine remains in the body of the subject for a long time after the administration, and continues to slowly produce the encoded proteins. Thus, excessive immune responses can be avoided. DNA vaccines can also be modified using a genetic engineering techniques. Optionally, such nucleic acids further encode a signal peptide and can be expressed with the signal peptide linked to peptide. Coding sequences of nucleic acids can be operably linked with regulatory sequences to ensure expression of the coding sequences, such as a promoter, enhancer, ribosome binding site, transcription termination signal, and the like. The nucleic acids encoding alpha-synuclein can occur in isolated form or can be cloned into one or more vectors. The nucleic acids can be synthesized by, for example, solid state synthesis or PCR of overlapping oligonucleotides. Nucleic acids encoding alpha-synuclein peptide or alpha-synuclein polypeptides with and without linkers and/or cleavable linkers and with or without protein-based carriers can be joined as one contiguous nucleic acid, e.g., within an expression vector.

DNA is more stable than RNA but involves some potential safety risks such as induction of anti-DNA antibodies, thus in some embodiments, the nucleic acid can be RNA. RNA nucleic acid that encodes an immunogen and is used as a vaccine can be referred to as a “RNA immunogen” or “RNA vaccine” or “mRNA vaccine” as the encoded polypeptides are expressed in vivo after administration of the RNA. Ribonucleic acid (RNA) vaccines can safely direct a subject’s cellular machinery to produce one or more (poly)peptide(s) of interest. In some embodiments, a RNA vaccine can be a non-replicating mRNA (messenger-RNA) or a virally derived, self-amplifying RNA. mRNA-based vaccines encode the antigens of interest and contain 5′ and 3′ untranslated regions (UTRs), whereas self-amplifying RNAs encode not only the antigens, but also the viral replication machinery that enables intracellular RNA amplification and abundant protein expression. In vitro transcribed mRNA can be produced from a linear DNA template using a T7, a T3 or an Sp6 phage RNA polymerase. The resulting product can contain an open reading frame that encodes the peptides of interest as disclosed herein, flanking 5′- and 3′-UTR sequences, a 5′ cap and a poly(A) tail. In some embodiments, a RNA vaccine can comprise trans-amplifying RNA (for example, see Beissert et al., Molecular Therapy January 2020 28(1):119-128). In certain embodiments, RNA vaccines encode an alpha-synuclein (poly)peptide as disclosed herein and are capable of expressing the alpha-synuclein peptides, in particular, if transferred into a cell such as an immature antigen presenting cell. RNA may also contain sequences which encode other polypeptide sequences such as immune stimulating elements. In some embodiments, the RNA of a RNA vaccine can be modified RNA. The term “modified” in the context of the RNA can include any modification of RNA which is not naturally present in RNA. For example, modified RNA can refer to RNA with a 5′-cap; however, RNA may comprise further modifications. A 5′-cap can be modified to possess the ability to stabilize RNA when attached thereto. In certain embodiments, a further modification may be an extension or truncation of the naturally occurring poly(A) tail or an alteration of the 5′- or 3′-untranslated regions (UTR). In some embodiments, the RNA e.g. or mRNA vaccine is formulated in an effective amount to produce an antigen specific immune response in a subject. For example, the RNA vaccine formulation is administered to a subject in order to stimulate the humoral and/or cellular immune system of the subject against the alpha-synuclein antigens, and thus may further comprise one or more adjuvant(s), diluents, carriers, and/or excipients, and is applied to the subject in any suitable route in order to elicit a protective and/or therapeutic immune reaction against the alpha-synuclein (poly)peptide antigens.

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J D et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

Techniques for the manipulation of nucleic acids, such as, e.g., generating mutations in sequences, sub-cloning, labeling probes, sequencing, hybridization and the like are well described in the scientific and patent literature. See, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immunofluorescence assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

### Pharmaceutical Compositions

Each of the peptides and immunogens described herein can be presented in a pharmaceutical composition that is administered with pharmaceutically acceptable adjuvants and pharmaceutically acceptable excipients. The adjuvant increases the titer of induced antibodies and/or the binding affinity of induced antibodies relative to the situation if the peptide were used alone. A variety of adjuvants can be used in combination with an immunogen of the disclosure to elicit an immune response. Some adjuvants augment the intrinsic response to an immunogen without causing conformational changes in the immunogen that affect the qualitative form of the response. An adjuvant may be a natural compound, a modified version of or derivative of a natural compound, or a synthetic compound.

Some adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). As used herein, MPL refers to natural and synthetic versions of MPL. Examples of synthetic versions include PHAD®, 3D-PHAD® and 3D(6A)-PHAD® (Avanti Polar Lipids, Alabaster, Alabama).

QS-21 is a triterpene glycoside or saponin isolated from the bark of the Quillaja saponaria Molina tree found in South America (see Kensil et al., in Vaccine Design: The Subunit and Adjuvant Approach (eds. Powell & Newman, Plenum Press, NY, 1995)) QS-21 products include Stimulon® (Antigenics, Inc., New York, NY; now Agenus, Inc. Lexington, MA) and QS-21 Vaccine Adjuvant (Desert King, San Diego, CA). QS-21 has been disclosed, characterized, and evaluated in US 5,057,540, and US 8,034,348, the disclosures of which are herein incorporated by reference. Additionally, QS-21 has been evaluated in numerous clinical trials in various dosages. See, NCT00960531 (clinicaltrials.gov/ct2/show/study/NCT00960531), Hüll et al., Curr Alzheimer Res. 2017 Jul; 14(7): 696-708 (evaluated 50 mcg of QS-21 in with various doses of vaccine ACC-001); Gilman et al., “Clinical effects of Abeta immunization (AN1792) in patients with AD in an interrupted trial” Neurology. 2005 May 10; 64(9):1553-62; Wald et al., “Safety and immunogenicity of long HSV-2 peptides complexed with rhHsc70 in HSV-2 seropositive persons” Vaccine 2011; 29(47):8520-8529; and Cunningham et al., “Efficacy of the Herpes Zoster Subunit Vaccine in Adults 70 Years of Age or Older.” NEJM. 2016 Sep 15; 375(11):1019-32. QS-21 is used in FDA approved vaccines including SHINGRIX. SHINGRIX contains 50 mcg of QS-21. In certain embodiments, the amount of QS-21 is from about 10 µg to about 500 µg.

TQL1055 is an analogue of QS-21 (Adjuvance Technologies, Lincoln, NE). The semi-synthetic TQL1055 has been characterized in comparison to QS-21 as having high purity, increased stability, decreased local tolerability, decreased systemic tolerability. TQL1055 has been disclosed, characterized, and evaluated in US20180327436 A1, WO2018191598 A1, WO2018200656 A1, and WO2019079160 A1, the disclosures of which are herein incorporated by reference. US20180327436 A1 teaches that 2.5 fold more TQ1055 was superior to 20 µg QS-21 but there was not an improvement over 50 µg TQ1055. However, unlike QS-21 there was no increase in either weight loss or hemolysis of RBC as the TQL1055 dose increased. WO2018200656 A1 teaches that with an optimal amount of TQ1055, one can lower the amount of antigen and achieve superior titers. In certain embodiments, the amount of TQL1055 is from about 10 µg to about 500 µg.

Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween 80. Ribi also contains refined mycobacterial products which act as immunostimulants and bacterial monophosphoryl lipid A. Other adjuvants can be CpG oligonucleotides (see WO 98/40100), cytokines (e.g., IL-1, IL-1 alpha and β peptides, IL-2, γ-INF, IL-10, GM-CSF), chemokines (e.g., MIP1-α and β, and RANTES), saponins, RNA, and/or TLR agonists (for example, TLR4 agonists such as MPL and synthetic MPL molecules), aminoalkyl glucosaminide phosphate and other TLR agonists. Adjuvants can be administered as a component of a therapeutic composition with an active agent or can be administered separately, before, concurrently with, or after administration of the therapeutic agent.

In various embodiments of the disclosure, the adjuvant is QS-21 (Stimulon™). In some compositions, the adjuvant is MPL. In certain embodiments, the amount of MPL is from about 10 µg to about 500 µg. In some compositions, the adjuvant is TQL1055. In certain embodiments, the amount of TQL1055 is from about 10 µg to about 500 µg. In some compositions, the adjuvant is QS21. In certain embodiments, the amount of QS21 is from about 10 µg to about 500 µg. In some compositions, the adjuvant is a combination of MPL and QS-21. In some compositions, the adjuvant is a combination of MPL and TQL1055. In some compositions, the adjuvant can be in a liposomal formulation.

In addition, some embodiments of the disclosure can comprise a multiple antigen presenting system (MAP). Multiple antigen-presenting peptide vaccine systems have been developed to avoid the adverse effects associated with conventional vaccines (i.e., live-attenuated, killed or inactivated pathogens), carrier proteins and cytotoxic adjuvants. Two main approaches have been used to develop multiple antigen presenting peptide vaccine systems: (1) the addition of functional components, e.g., T-cell epitopes, cell-penetrating peptides, and lipophilic moieties; and (2) synthetic approaches using size-defined nanomaterials, e.g., self-assembling peptides, non-peptidic dendrimers, and gold nanoparticles, as antigen-displaying platforms. Use of a multiple antigenic peptide (MAP) system can improve the sometimes poor immunogenicity of subunit peptide vaccines. In a MAP system, multiple copies of antigenic peptides are simultaneously bound to the a- and ε-amino groups of a non-immunogenic Lys-based dendritic scaffold, helping to confer stability from degradation, thus enhancing molecular recognition by immune cells, and induction of stronger immune responses compared with small antigenic peptides alone. In some compositions, the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the peptides of the disclosure can be formulated in aqueous solutions, preferably in physiologically compatible buffers such as Hank’s solution, Ringer’s solution, or physiological saline or acetate buffer (to reduce discomfort at the site of injection). The solution can contain formulatory agents such as suspending, stabilizing and/or dispersing agents. Alternatively, peptide compositions can be in lyophilized form for constitution with a suitable vehicle, e.g., sterile pyrogen-free water, before use.

Peptides and polypeptides as described herein (and optionally a carrier fused to the peptide(s)) can also be administered in the form of a nucleic acid encoding the peptide(s) and expressed in situ in a subject. A nucleic acid segment encoding an immunogen is typically linked to regulatory elements, such as a promoter and enhancer that allow expression of the DNA segment in the intended target cells of a subject. For expression in blood cells, as is desirable for induction of an immune response, promoter and enhancer elements from, for example, light or heavy chain immunoglobulin genes or the CMV major intermediate early promoter and enhancer are suitable to direct expression. The linked regulatory elements and coding sequences are often cloned into a vector.

DNA and RNA can be delivered in naked form (i.e., without colloidal or encapsulating materials). Alternatively a number of viral vector systems can be used including retroviral systems (see, e.g., Boris-Lawrie and Teumin, Cur. Opin. Genet. Develop. 3(1):102-109 (1993)); adenoviral vectors (see, e.g., Bett et al, J. Virol. 67(10);5911-21 (1993)); adeno-associated virus vectors (see, e.g., Zhou et al., J. Exp. Med. 179(6):1867-75 (1994)), viral vectors from the pox family including vaccinia virus and the avian pox viruses, viral vectors from the alpha virus genus such as those derived from Sindbis and Semliki Forest Viruses (see, e.g., Dubensky et al., J. Virol. 70(1):508-519 (1996)), Venezuelan equine encephalitis virus (see US 5,643,576) and rhabdoviruses, such as vesicular stomatitis virus (see WO 96/34625) and papillomaviruses (WO 94/12629; Ohe et al., Human Gene Therapy 6(3):325-333 (1995); and Xiao & Brandsma, Nucleic Acids. Res. 24(13):2620-2622 (1996)).

DNA and RNA encoding an immunogen, or a vector containing the same, can be packaged into liposomes, nanoparticles or lipoproteins complexes. Other suitable polymers, include, for example, protamine liposomes, polysaccharide particles, cationic nanoemulsion, cationic polymer, cationic polymer liposome, cationic lipid nanoparticles, cationic lipid, cholesterol nanoparticles, cationic lipid-cholesterol, PEG nanoparticle, or dendrimer nanoparticles. Additional suitable lipids and related analogs are described by US 5,208,036, US 5,264,618, US 5,279,833, and US 5,283,185, each of which are herein incorporated by reference in their entirety. Vectors and DNA encoding an immunogen can also be adsorbed to or associated with particulate carriers, examples of which include polymethyl methacrylate polymers and polylactides and poly(lactide-co-glycolides), (see, e.g., McGee et al., J. Micro Encap. Mar-April 1997; 14(2):197-210).

Pharmaceutically acceptable carrier compositions can also include additives, including, but not limited to, water, pharmaceutically acceptable organic solvents, collagen, polyvinyl alcohol, polyvinylpyrrolidone, carboxyvinyl polymers, carboxymethylcellulose sodium, sodium polyacrylate, sodium alginate, water-soluble dextran, carboxymethyl starch sodium, pectin, methylcellulose, ethylcellulose, xanthan gum, gum arabic, casein, agar, polyethylene glycol, diglycerine, glycerine, propylene glycol, petrolatum, paraffin, stearyl alcohol, stearic acid, human serum albumin, mannitol, sorbitol, lactose, and surfactants acceptable as pharmaceutical additives.

### Subjects Amenable to Treatment

The presence of abnormally aggregated intracellular alpha-synuclein, has been found in several diseases including Parkinson’s disease (PD), Parkinson’s disease with dementia (PDD), dementia with Lewy bodies (DLB), multiple system atrophy (MSA; including the MSA-parkinsonian (MSA-P) variant and the MSA-cerebellar (MSA-C) variant), Alzheimer’s disease (AD); including the Lewy body variant of AD, and other variants that comprise alpha-synuclein-related pathology), and neurodegeneration with brain iron accumulation (NBIA).

The compositions and methods of the disclosure can be used in treatment or prophylaxis of any of these diseases and other synucleinopathies. Because of the widespread association between neurological diseases and alpha-synuclein, the compositions and methods of the disclosure can be used in treatment or prophylaxis of any subject showing elevated levels of alpha-synuclein (e.g., in the CSF) compared with a mean value in individuals without neurological disease. The compositions and methods of the disclosure can also be used in treatment or prophylaxis of neurological disease in individuals having a mutation in alpha-synuclein associated with neurological disease. The methods are particularly suitable for treatment or prophylaxis of alpha-synucleinopathies.

Subjects amenable to treatment include individuals at risk of disease but not showing symptoms, as well as patients presently showing symptoms, including treatment naïve subjects that have not been previous treated for disease. Subjects at risk of disease include those in an aging population, asymptomatic subjects with alpha-synuclein pathologies and having a known genetic risk of disease. Such individuals include those having relatives who have experienced this disease, and those whose risk is determined by analysis of genetic or biochemical markers. Genetic markers of risk include mutations in alpha-synuclein, as well as mutations in other genes associated with neurological disease. For example, the ApoE4 allele in heterozygous and even more so in homozygous form is associated with risk of Alzheimer’s disease (AD). Other markers of risk of Alzheimer’s disease include mutations in the APP gene, particularly mutations at position 717 and positions 670 and 671 referred to as the Hardy and Swedish mutations respectively, mutations in the presenilin genes, PS1 and PS2, a family history of AD, hypercholesterolemia or atherosclerosis. Individuals presently suffering from Alzheimer’s disease, Parkinson’s disease and other neurological disorders involving alpha-synuclein can be recognized by PET imaging, some (e.g., Alzheimer’s disease) from characteristic dementia, as well as the presence of risk factors described above. In addition, a number of diagnostic tests are available for identifying individuals who have, e.g., Parkinson’s disease or Alzheimer’s disease. For Alzheimer’s disease, these include measurement of CSF or blood alpha-synuclein or phospho-alpha-synuclein and Aβ42 levels. Elevated alpha-synuclein or phospho-alpha-synuclein and decreased Aβ42 levels signify the presence of AD. Mutations that are associated with Parkinson’s disease include, for example, Ala30Pro or Ala53Thr, or mutations in other genes such as leucine-rich repeat kinase (LRRK2 or PARK8). Subjects can also be diagnosed with any of the neurological diseases mentioned above by the criteria of the DSM IV TR.

In asymptomatic subjects, treatment can begin at any age (e.g., 10, 20, 30, or more). Usually, however, it is not necessary to begin treatment until a subject reaches 20, 30, 40, 50, 60, 70, 80, or 90 years of age. Treatment typically entails multiple dosages over a period of time. Treatment can be monitored by assaying antibody levels over time. If the response falls, a booster dosage is indicated. In the case of potential Down’s syndrome patients, treatment can begin antenatally by administering therapeutic agent to the mother or shortly after birth.

### Methods of Treatments and Uses

The disclosure provides methods of inhibiting or reducing aggregation of alpha-synuclein in a subject having or at risk of developing a neurodegenerative disease (e.g., Parkinson’s disease, Alzheimer’s disease, dementia with Lewy bodies (DLB), and the like). The methods include administering to the subject the compositions as disclosed herein. A therapeutically effective amount is a dosage that, when given for an effective period of time, achieves the desired immunological or clinical effect. Dosage regimens may be adjusted to provide the optimum therapeutic response. For example, several divided doses may be administered at set intervals (e.g., weekly, monthly) or the dose may be proportionally reduced as indicated by the exigencies of the therapeutic situation.

In prophylactic applications, the compositions described herein can be administered to a subject susceptible to, or otherwise at risk of a disease (e.g., Parkinson’s disease, dementia with Lewy bodies (DLB), Alzheimer’s disease, and the like) in a regimen (dose, frequency and route of administration) effective to reduce the risk, lessen the severity, or delay the onset of at least one sign or symptom of the disease. In particular, the regimen is effective to inhibit or delay aggregation of alpha-synuclein or phospho-alpha-synuclein (and paired filaments formed from them) in the brain, and/or inhibit or delay its toxic effects and/or inhibit/or delay development of behavioral deficits. In therapeutic applications, the compositions described herein are administered to a subject suspected of, or a patient already suffering from a disease (e.g., Parkinson’s disease, dementia with Lewy bodies (DLB), Alzheimer’s disease, and the like) in a regimen (dose, frequency and route of administration) effective to ameliorate or at least inhibit further deterioration of at least one sign or symptom of the disease. In particular, the regimen is preferably effective to reduce or at least inhibit further increase of levels of alpha-synuclein, phospho-alpha-synuclein, or paired filaments formed from them, associated toxicities and/or behavioral deficits.

A regimen is considered therapeutically or prophylactically effective if an individual treated achieves an outcome more favorable than the mean outcome in a control population of comparable subjects not treated by methods of the invention, or if a more favorable outcome is demonstrated in treated subjects versus control subjects in a controlled clinical trial (e.g., a phase II, phase II/III or phase III trial) at the p < 0.05 or 0.01 or even 0.001 level.

Effective doses of vary depending on many different factors, such as means of administration, target site, physiological state of the patient, whether the patient is an ApoE carrier, whether the patient is human or an animal, other medications administered, and whether treatment is prophylactic or therapeutic.

In some embodiments, the effective amount is a total dose of 25 µg to 1000 µg, or 50 µg to 1000 µg. In some embodiments, the effective amount is a total dose of 100 µg. In some embodiments, the effective amount is a dose of 25 µg administered to the subject a total of two times. In some embodiments, the effective amount is a dose of 100 µg administered to the subject a total of two times. In some embodiments, the effective amount is a dose of 400 µg administered to the subject a total of two times. In some embodiments, the effective amount is a dose of 500 µg administered to the subject a total of two times. In some embodiments, a RNA (e.g., mRNA) vaccine is administered to a subject by intradermal, intramuscular injection, or by intranasal administration.

In some embodiments, the amount of an agent for active immunotherapy varies from 1 to 1,000 micrograms (µg), or from 0.1-500 µg, or from 10 to 500 µg, or from 50 to 250 µg per patient and can be from 1-100 or 1-10 µg per injection for human administration. The timing of injections can vary significantly from once a day, to once a week, to once a month, to once a year, to once a decade. A typical regimen consists of an immunization followed by booster injections at time intervals, such as 6 week intervals or two months. Another regimen consists of an immunization followed by one or more booster injections 1, 2, 3, 4, 5, 6, 7, 8, 9, 10, 11, or 12 months later. Another regimen entails an injection every two months for life. Alternatively, booster injections can be on an irregular basis as indicated by monitoring of immune response. The frequency of administration may be once or more as long as the side effects are within a clinically acceptable range.

In some embodiments, the compositions or methods as disclosed herein comprise administering to a subject a nucleic acid vaccine comprising one or more DNA or RNA polynucleotides having an open reading frame encoding a first peptide and a second peptide wherein a dosage of between 10 µg/kg and 400 µg /kg of the nucleic acid vaccine is administered to the subject. In some embodiments the dosage of the RNA polynucleotide is 1-5 µg, 5-10 µg, 10-15 µg, 15-20 µg, 10-25 µg, 20-25 µg, 20-50 µg, 30-50 µg, 40-50 µg, 40-60 µg, 60-80 µg, 60-100 µg, 50-100 µg, 80-120 µg, 40-120 µg, 40-150 µg, 50-150 µg, 50-200 µg, 80-200 µg, 100-200 µg, 120-250 µg, 150-250 µg, 180-280 µg, 200-300 µg, 50-300 µg, 80-300 µg, 100-300 µg, 40-300 µg, 50-350 µg, 100-350 µg, 200-350 µg, 300-350 µg, 320-400 µg, 40-380 µg, 40-100 µg, 100-400 µg, 200-400 µg, or 300-400 µg per dose. In some embodiments, the nucleic acid is administered to the subject by intradermal or intramuscular injection. In some embodiments, the nucleic acid is administered to the subject on day zero. In some embodiments, a second dose of the nucleic acid is administered to the subject on day seven, or fourteen, or twenty one.

The compositions described herein are preferably administered via a peripheral route (i.e., one in which the administered composition results in a robust immune response and/or the induced antibody population crosses the blood brain barrier to reach an intended site in the brain, spinal cord, or eye). For peripheral diseases, the induced antibodies leave the vasculature to reach the intended peripheral organs. Routes of administration include oral, subcutaneous, intranasal, intradermal, or intramuscular. Some routes for active immunization are subcutaneous and intramuscular. Intramuscular administration and subcutaneous administration can be made at a single site or multiple sites. Intramuscular injection is most typically performed in the arm or leg muscles. In some methods, agents are injected directly into a particular tissue where deposits have accumulated.

The number of dosages administered can be adjusted to result in a more robust immune response (for example, higher titers). For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. For chronic disorders, a vaccine/immunotherapy as disclosed herein can be administered at regular intervals, e.g., weekly, fortnightly, monthly, quarterly, every six months for at least 1, 5 or 10 years, or the life of the patient.

An effective amount of a DNA or RNA encoded immunogen can be between about 1 nanogram and about 1 gram per kilogram of body weight of the recipient, or about between about 0.1 µg/kg and about 10 mg/kg, or about between about 1 µg/kg and about 1 mg/kg. Dosage forms suitable for internal administration preferably contain (for the latter dose range) from about 0.1 µg to 100 µg of active ingredient per unit. The active ingredient may vary from 0.5 to 95% by weight based on the total weight of the composition. Alternatively, an effective dose of dendritic cells loaded with the antigen is between about 10⁴ and 10⁸ cells. Those skilled in the art of immunotherapy will be able to adjust these doses without undue experimentation.

The nucleic acid compositions may be administered in a convenient manner, e.g., injection by a convenient and effective route. Routes can include, but are not limited to, intradermal “gene gun” delivery or intramuscular injection. The modified dendritic cells are administered by subcutaneous, intravenous or intramuscular routes. Other possible routes include oral administration, intrathecal, inhalation, transdermal application, or rectal administration.

Depending on the route of administration, the composition may be coated in a material to protect the compound from the action of enzymes, acids and other natural conditions which may inactivate the compound. Thus, it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol) or in an appropriate carrier such as liposomes (including water-in-oil-in-water emulsions) as well as conventional liposomes (Strejan et al., J. Neuroimmunol 7(1):27-41, 1984).

The immunotherapeutic compositions disclosed herein may also be used in combination with other treatments for diseases associated with the accumulation and/or aggregation of alpha-synuclein, for example, anti-alpha-synuclein antibodies such as antibodies that specifically bind to any of the alpha-synuclein epitopes disclosed herein, ABBV-8E12, gosuranemab, zagotenemab, RG-6100, BIIB076 or any of the antibodies disclosed in WO2014/165271, US 10,501,531, WO2017/191559, WO2017/191560, WO2017/191561, US 20190330314, US 20190330316, and WO2018/204546. In some combination therapy methods, the patient receives passive immunotherapy prior to the active immunotherapy methods disclosed herein. In other methods, the patient receives passive and active immunotherapy during the same period of treatment. Alternatively, patients may receive active immunotherapy prior to passive immunotherapy. Combinations may also include small molecule therapies and non-immunogenic therapies such as RAZADYNE® (galantamine), EXELON® (rivastigmine), and ARICEPT® (donepezil) and other compositions that improve the function of nerve cells in the brain.

The compositions of the disclosure may be used in the manufacture of medicaments for the treatment regimens described herein.

### Treatment Regimens

Desired outcomes of the methods of treatment as disclosed herein vary according to the disease and patient profile and are determinable to those skilled in the art. Desired outcomes include an improvement in the patient’s health status. Generally, desired outcomes include measurable indices such as reduction or clearance of pathologic alpha synuclein aggregates, decreased or inhibited alpha synuclein aggregation, and increased immune response to pathologic and/or aggregated alpha synuclein aggregates. Desired outcomes also include amelioration of alpha-synucleinopathic disease-specific symptoms. As used herein, relative terms such as “improve,” “increase,” or “reduce” indicate values relative to a control, such as a measurement in the same individual prior to initiation of treatment described herein, or a measurement in a control individual or group. A control individual is an individual afflicted with the same alpha synucleinopathic disease as the individual being treated, who is about the same age as the individual being treated (to ensure that the stages of the disease in the treated individual and the control individual are comparable), but who has not received treatment using the disclosed immunotherapy/vaccine formulations. Alternatively, a control individual is a healthy individual, who is about the same age as the individual being treated. Changes or improvements in response to therapy are generally statistically significant and described by a p-value less than or equal to 0.1, less than 0.05, less than 0.01, less than 0.005, or less than 0.001 may be regarded as significant.

Effective doses of the compositions as disclosed herein, for the treatment of a subject vary depending upon many different factors, including means of administration, target site, physiological state of the patient, whether the patient is human or an animal, other medications administered, if any, and whether treatment is prophylactic or therapeutic. Treatment dosages can be titrated to optimize safety and efficacy. The amount of immunogen can also depend on whether adjuvant is also administered, with higher dosages being required in the absence of adjuvant. The amount of an immunogen for administration sometimes varies from 1-500 µg per patient and more usually from 5-500 µg per injection for human administration. Occasionally, a higher dose of 1-2 mg per dosage is used. Typically, about 10, 20, 50 or 100 µg is used for each human dosage. The timing of dosages can vary significantly from once a day, to once a year, to once a decade. On any given day that a dosage of immunogen is given, the dosage is greater than 1 µg/patient and usually greater than 10 µg/patient if adjuvant is also administered, and greater than 10 µg/patient and usually greater than 100 µg/patient in the absence of adjuvant. A typical regimen consists of an immunization followed by booster dosage(s) at 6-week intervals. Another regimen consists of an immunization followed by booster dosage(s) 1, 2, 3, 4, 5, 6, 7, 8, 9, 10, 11,or 12 months later. Another regimen entails dosage(s) every two months for life. Alternatively, booster dosage(s) can be on an irregular basis as indicated by monitoring of immune response.

When administered in combination with a second treatment, e.g., for Alzheimer’s disease or Parkinson’s disease with dementia (PDD), such as Razadyne® (galantamine), Exelon® (rivastigmine), and Aricept® (donepezil), the second treatment can be administered according the product label or as necessary in view of the treatment with the compositions of the disclosure.

### Kits

The disclosure further provides kits (e.g., containers) comprising the compositions disclosed herein and related materials, such as instructions for use (e.g., package insert). The instructions for use may contain, for example, instructions for administration of the compositions and optionally one or more additional agents. The containers of peptide and/or nucleic acid compositions may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products. Kits can also include a second container comprising a pharmaceutically acceptable buffer, such as bacteriostatic water for injection (BWFI), phosphate-buffered saline, Ringer’s solution and dextrose solution. It can also include other materials desirable from a commercial and user standpoint, including other buffers, diluents, filters, needles, and syringes.

### Uses

Each of the peptides, polypeptides, immunogens, and pharmaceutical compositions described herein may be for use in treating one or more of the diseases as described herein. In addition, each of the peptides, polypeptides, immunogens, and pharmaceutical compositions described herein may be for use in methods for treating one or more of the diseases as described herein. Each of the peptides, polypeptides, immunogens, and pharmaceutical compositions described herein may be used in a method for manufacturing a medicament for treating or use in treating one or more of the diseases as described herein.

The following are provided for exemplification purposes only and are not intended to limit the scope of the invention described in broad terms above.

All U.S. and international patent applications identified herein are incorporated by reference in their entirety.

### EXAMPLES

### Example 1: Animal Immunizations

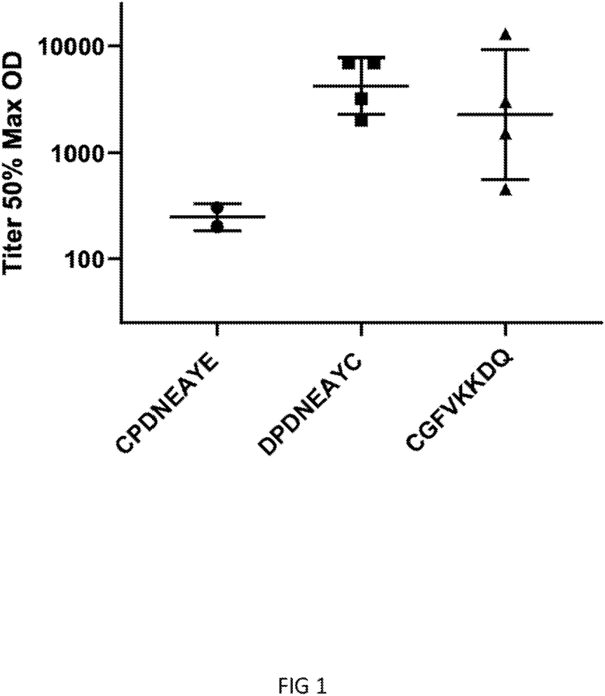

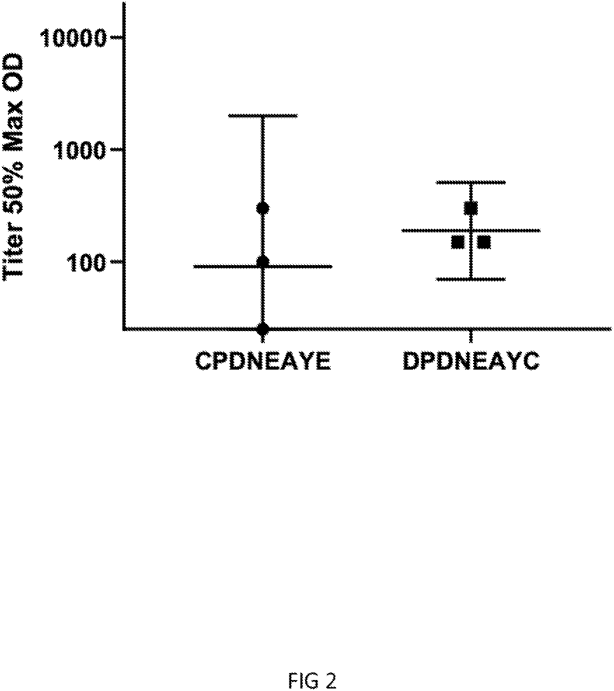

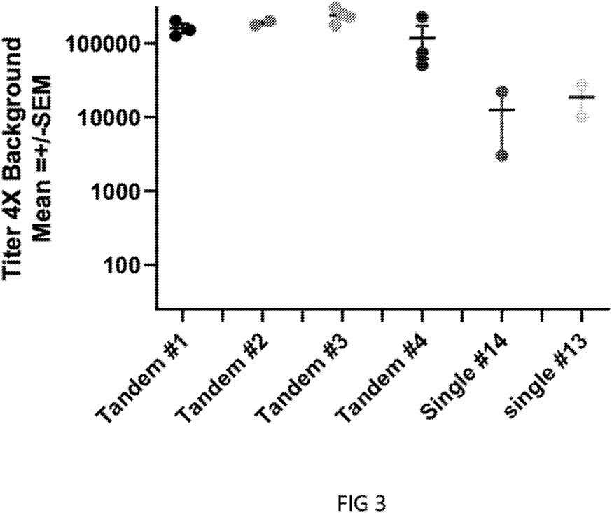

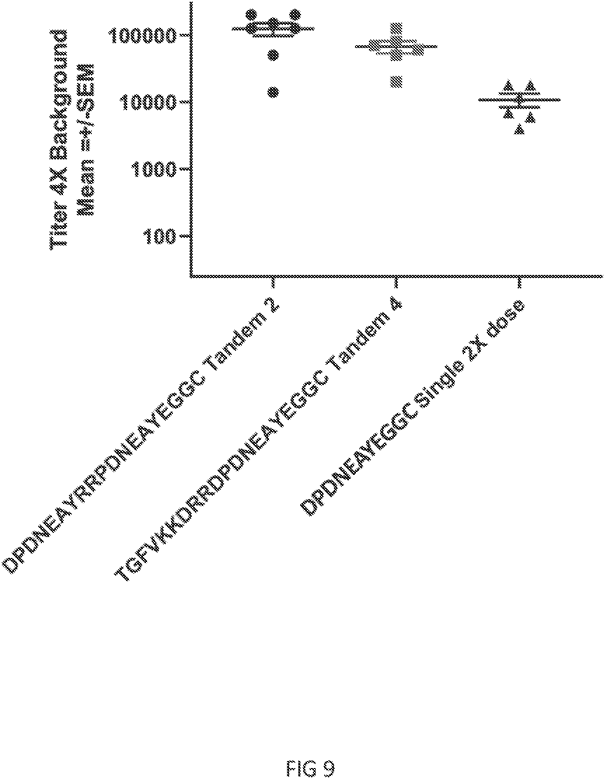

For experiments shown in FIG. 1 and FIG. 2, female Swiss Webster mice were injected subcutaneously at two sites with 100 µl of test article on day 0, 14, 42 and 70. For experiments shown in FIG. 3 to FIG. 8, female Swiss Webster mice were injected subcutaneously at two sites with 100 µl of test article on day 0 and week 4 and week 8. For the experiments shown in FIG. 9 to FIG. 11, female B6 mice were injected subcutaneously at two sites with 100 µl of test article on day 0 and week 4 and week 8. Test article was prepared by combining 25 µg of test immunogen and 25 µg of QS21 adjuvant in 200 µl phosphate buffered saline (PBS) except for the experiments shown in FIG. 9 to FIG. 11, in which 2x (50 µg) of test immunogen (SEQ ID NO:77) was used. Mice used in the experiments shown in FIG. 1 and FIG. 2 were bled on day 21, 49 and 77 by nicking tails and collecting 50 µl of blood, followed by processing to serum. Mice used in the experiments shown in FIG. 3 to FIG. 11 were bled at week 5 and week 8. The peptides tested included CPDNEAYE (SEQ ID NO:73), DPDNEAYC (SEQ ID NO:74), CGFVKKDQ (SEQ ID NO:75), PDNEAYEGGC (SEQ ID NO:76), DPDNEAYEGGC (SEQ ID NO:77), PDNEAYERRDPDNEAYGGC (SEQ ID NO:78), DPDNEAYRRPDNEAYEGGC (SEQ ID NO:79), PDNEAYERRTGFVKKDGGC (SEQ ID NO:80), or TGFVKKDRRDPDNEAYEGGC (SEQ ID NO:81). Immunogens contained one alpha-synuclein (poly)peptide, a C-terminal linker and a C-terminal cysteine (i.e., -Gly-Gly-Cys-; specifically noted in SEQ ID NO:76 through SEQ ID NO:81 by a C-terminal “-GGC” but not for SEQ ID NO:73 to SEQ ID NO:75) and were coupled through the C-terminal cysteine to CRM-197 with a maleimide linkage.

Guinea pigs were injected intramuscularly with 50 µg of a test immunogen, 25 µg QS21 in 200 µl of Addavax on day 0, 21, 49 and 77. Bleeds were done 7 days post immunization. The peptides tested included CPDNEAYE (SEQ ID NO:73) and DPDNEAYC (SEQ ID NO:74). Immunogens contained one alpha-synuclein peptide, a C-terminal linker. and a C-terminal cysteine (i.e., -Gly-Gly-Cys-) and were coupled through the C-terminal cysteine to CRM-197 with a maleimide linkage.

Female Guinea pigs were at least 5 weeks old at the start of the study having an approximate body weight of 350-500 g. Appropriate animal housing and research procedures for animal husbandry and care were conducted in an accredited facility in accordance with the guidelines of the U.S. Department of Agriculture’s (USDA) and the Assessment and Accreditation of Laboratory Animal Care (AAALAC) International.

The immunogen concentration was 0.5 mg/ml. Prior to each administration of the test immunogen, approximately a 3 cm² area on each hind limb was shaved and wiped with ethanol for visualization of the injection site. Each animal received a test immunogen dose of 200 microliters (0.25 micrograms/microliter) divided into two separate sites each of 100 microliter per injection (i.e., animals received 50 µg of immunogen in 100 µl PBS + 25 µg of QS-21 in 100 µl Addavax). A 25G-27G needle was inserted intramuscularly into the hind limb, approximately 0.25 - 0.5 cm deep, and injected at 100 microliters per site. Injection sites were rotated each administration between four separate sites per hind limb and separated by at least 2 cm.

### Example 2: Measurement of Antibody Titers

Whole blood samples were collected into clot activator tubes via jugular vein at 250-350 microliters per collection at weeks 1, 4, 8 and 12 for Guinea pigs and 50 microliters per collection at weeks 1, 3, 7 and 11 by nicking tails for mice. The maximum volume of whole blood was collected into clot activator tubes via cardiac puncture at termination on the final collection week. All blood samples were allowed to clot at room temperature for greater than 30 minutes, centrifuged at ambient temperature (approximately 20-25° C.) at 3,000 RPM for 10-15 minutes, and serum supernatant was transferred individually into clean cryovials. Serum supernatant was stored frozen at -80° C. (± 12° C.).

### Titer Guinea Pig Bleed - Alpha-Synuclein Peptides

2 µg/ml alpha-synuclein was coated at coated on to the plate 100 µl per well in PBS and incubated overnight at room temperature. Plates were blocked for 1 hour with 1% BSA in PBS. Plates were aspirated, and to row A 200 µl of 0.1% BSA in PBS Tween was added. In column 1 negative Guinea pig serum was added at 1/100 dilution while the rest of the row contained 1/100 test serums. Rows were serially diluted by 50% per step down the plate giving dilution range of 1/100 to 1/12800. Wells were incubated for 2 hours at room temperature then were washed. A 1/5000 dilution of anti-Guinea pig IgG HRP in 0.1% BSA in PBS Tween was prepared and then 100 µl added to the washed well. Samples were incubated for 1 hour and then washed. OPD substrate was prepared using Thermo-Fisher OPD tablets at 1 tablet per 10 mL. Thermo-Fisher substrate buffer was added at 1/10 and each well had 100 µl added and was incubated for 15 minutes. 50 µl of 2N H₂SO₄ was added to stop the reaction and plates were read at 490 nm on a Molecular Devices Spectromax. Titer was defined as the dilution giving 50% maximum OD and was extrapolated if it fell between dilutions.

### Titers on Alpha-Synuclein Mouse

2 µg/ml recombinant alpha-synuclein was coated on to the plate 100 µl per well in PBS and incubated overnight at room temperature. Plates were blocked for 1 hour with 1% BSA in PBS. Plates were aspirated, and to row A 200 µl of 0.1% BSA in PBS Tween was added. In column 1, negative mouse serum was added at 1/100 while the rest of the row contained 1/100 test sera. Rows were serially diluted ½ down the plate giving dilution of 1/100 to 1/12800. Wells were incubated 2 hours at room temperature then were washed. A 1/5000 dilution of anti-mouse IgG HRP in 0.1% BSA in PBS Tween was prepared and then 100 µl added to the washed well. The reaction mixture was incubated for 1 hour and then was washed. OPD substrate was prepared using Thermo-Fisher OPD tablets at 1 tablet per 10 mL. Thermo-Fisher substrate buffer was added at a 1/10 dilution (100 µl added to each well), and the mixture was incubated for 15 minutes. 50 µl of 2N H₂SO₄ was added to stop the reaction and plates were read at 490 nm on a Molecular Devices Spectromax. Titer was defined as the dilution giving 50% maximum OD measurement and was extrapolated if it fell between dilutions.

Antibody titers observed in Guinea pigs immunized as described above are shown in Table 1. Immunizations were conducted with QS21 in Addavax. The titers reported are for the bleed after the third injection. These results are represented in FIG. 1.

TABLE 1

Antibody titers in Guinea pigs (GP) immunized with alpha-synuclein epitopes

| Alpha-synuclein Epitope in immunogen | GP 1 Titer | GP 2 Titer | GP 3 Titer |
|---|---|---|---|
| CPDNEAYE (SEQ ID NO:73) | 100 | 25 | 300 |
| DPDNEAYC (SEQ ID NO:74) | 150 | 150 | 300 |

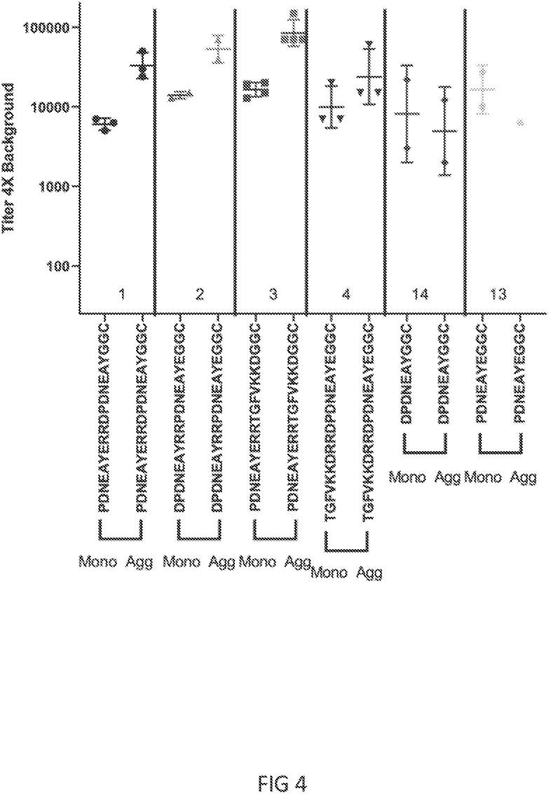

Antibody titers observed in mice immunized as described above are shown in Table 2, Table 3, Table 4 and Table 5. Immunizations were conducted with QS21. The titers reported are for the bleed after the third injection unless noted otherwise. Table 2 results are represented in FIG. 2; Table 3 results are represented in FIG. 3; Table 4 results are represented in FIG. 4; and Table 5 results are represented in FIG. 9.

TABLE 2

Antibody titers in mice immunized with alpha-synuclein epitopes

| Alpha-synuclein Epitope in immunogen | Mouse 1 Titer | Mouse 2 Titer | Mouse 3 Titer | Mouse 4 Titer |
|---|---|---|---|---|
| CPDNEAYE (SEQ ID NO:73) | 200 | | 300 | |
| DPDNEAYC (SEQ ID NO:74) | 2000 | 7000 | 3200 | 7000 |
| CGFVKKDQ (SEQ ID NO:75) | 3000 | 1500 | 450 | 13000 |

TABLE 3

Antibody titers in mice immunized with alpha-synuclein epitopes

| Sequences | SEQ ID NO | Mouse 1 titer | Mouse 2 titer | Mouse 3 titer | Mouse 4 titer |
|---|---|---|---|---|---|
| “Tandem #1” | 78 | dead | | | |
| Bleed 1 | | | 7000 | 5000 | 6300 |
| Bleed 2 | | | 200000 | 150000 | 125000 |
| “Tandem #2” | 79 | dead | | | |
| Bleed 1 | | | 13000 | 15000 | dead |
| Bleed 2 | | | 200000 | 175000 | |
| “Tandem #3” | 80 | | | | |
| Bleed 1 | | 12800 | 20000 | 19000 | 15000 |
| Bleed 2 | | 250000 | 300000 | 225000 | 175000 |
| “Tandem #4” | 81 | | | | |
| Bleed 1 | | 20000 | dead | 7000 | 7000 |
| Bleed 2 | | 225000 | | 75000 | 50000 |
| “Single #14” | 77 | | | | |
| Bleed 2 only (Bleed 1 different subs) | | 3000 | | 22000 | |
| Single #13″ | 76 | | | | |
| Bleed 2 only (Bleed 1 different subs) | | 10000 | | 27000 | |

TABLE 4

Antibody titers against monomeric or soluble aggregated alpha-synuclein in mice immunized with alpha-synuclein epitopes. Bleeds taken after the second injection

| SEQ ID NO (and column in FIG. 4 ) | Monomeric (M) or Soluble aggregated (A) Syn | mouse | titer |
|---|---|---|---|
| 78 (column “1”) | M | 1 | 7000 |
| 78 (column “1”) | M | 2 | 5000 |
| 78 (column “1”) | M | 3 | 6300 |
| 78 (column “1”) | A | 4 | 50000 |
| 78 (column “1”) | A | 5 | 30000 |
| 78 (column “1”) | A | 6 | 24000 |
| 79 (column “2”) | M | 1 | 13000 |
| 79 (column “2”) | M | 2 | 15000 |
| 79 (column “2”) | A | 3 | 70000 |
| 79 (column “2”) | A | 4 | 40000 |
| 80 (column “3”) | M | 1 | 12800 |
| 80 (column “3”) | M | 2 | 20000 |
| 80 (column “3”) | M | 3 | 19000 |
| 80 (column “3”) | M | 4 | 15000 |
| 80 (column “3”) | A | 5 | 70000 |
| 80 (column “3”) | A | 6 | 70000 |
| 80 (column “3”) | A | 7 | 70000 |
| 80 (column “3”) | A | 8 | 150000 |
| 81 (column “4”) | M | 1 | 20000 |
| 81 (column “4”) | M | 2 | 7000 |
| 81 (column “4”) | M | 3 | 7000 |
| 81 (column “4”) | A | 4 | 60000 |
| 81 (column “4”) | A | 5 | 15000 |
| 81 (column “4”) | A | 6 | 15000 |
| 77 (column “14”) | M | 1 | 3000 |
| 77 (column “14”) | M | 2 | 22000 |
| 77 (column “14”) | A | 3 | 2000 |
| 77 (column “14”) | A | 4 | 12200 |
| 76 (column “13”) | M | 1 | 10000 |
| 76 (column “13”) | M | 2 | 27000 |
| 76 (column “13”) | A | 3 | 6250 |

TABLE 5

Antibody titers in mice immunized with alpha-synuclein epitopes, including 2x dosing for monomeric alpha-synuclein peptide (SEQ ID NO:77) to compensate for having a single alpha-synuclein peptide compared to two for the tandem sequences

| Sample | SEQ ID NO | Mouse | Titer |
|---|---|---|---|
| “Tandem 2” | 79 | 1 | 200000 |
| “Tandem 2” | 79 | 2 | 14000 |
| “Tandem 2” | 79 | 3 | 150000 |
| “Tandem 2” | 79 | 4 | 200000 |
| “Tandem 2” | 79 | 5 | 125000 |
| “Tandem 2” | 79 | 6 | 125000 |
| “Tandem 2” | 79 | 7 | 50000 |
| “Tandem 4” | 81 | 8 | 70000 |
| “Tandem 4” | 81 | 9 | 125000 |
| “Tandem 4” | 81 | 10 | 20000 |
| “Tandem 4” | 81 | 11 | 50000 |
| “Tandem 4” | 81 | 12 | 80000 |
| “Tandem 4” | 81 | 13 | 60000 |
| “Single 2x dose” | 77 | 14 | 4000 |
| “Single 2x dose” | 77 | 15 | 7000 |
| “Single 2x dose” | 77 | 16 | 18000 |
| “Single 2x dose” | 77 | 17 | 6000 |
| “Single 2x dose” | 77 | 18 | 18000 |
| “Single 2x dose” | 77 | 19 | 12000 |

### Example 3: Staining of Parkinson’s Brain Tissue With Sera From Animals Immunized With a Vaccine as Disclosed Herein

Fresh frozen human brain tissues from autopsied Parkinson’s disease donors or non-diseased controls was embedded in optimal cutting temperature compound (OCT compound), and cut in a cryostat to generate 10 µm frozen sections. The tissue sections were incubated in a solution of glucose oxidase and beta D-glucose, in the presence of sodium azide, to block endogenous peroxidase. The staining with sera from vaccinated mice or control mice was then carried out at 1:1000 dilution, in an automated Leica Bond Rx Stainer (Leica Biosystems). Antibody binding was detected using the Bond Polymer Refine Detection Kit (DS9800, Leica Biosystems), which is based on an anti-mouse polymer detection, DAB visualization and hematoxylin nuclear counter-staining. After cover-slipping, the stained tissue slides were digitally imaged with a Hamamatsu NanoZoomer 2.0HT slide scanner (Hamamatsu Corporation) with an NDP.scan, 2.5.85 software. The digitized images were viewed and analyzed using the NDP.view, 2.7.43.0 software.

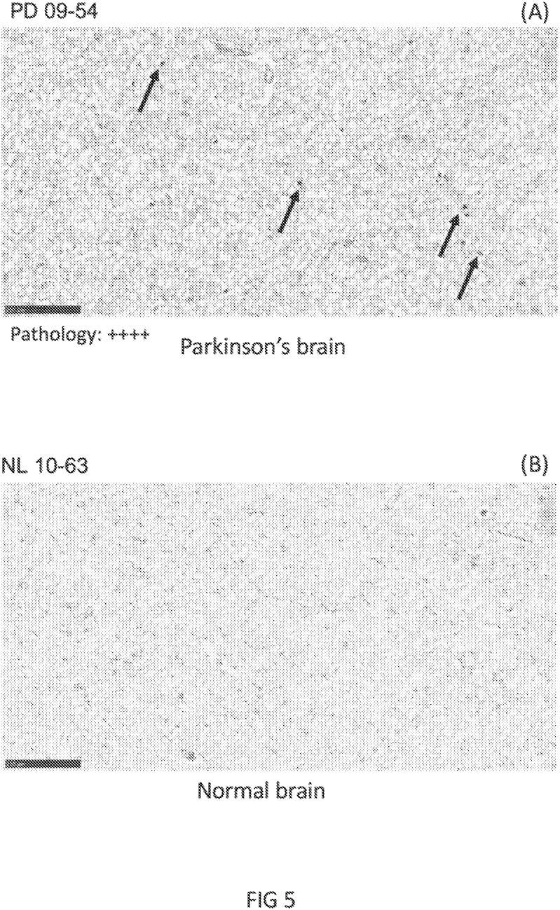

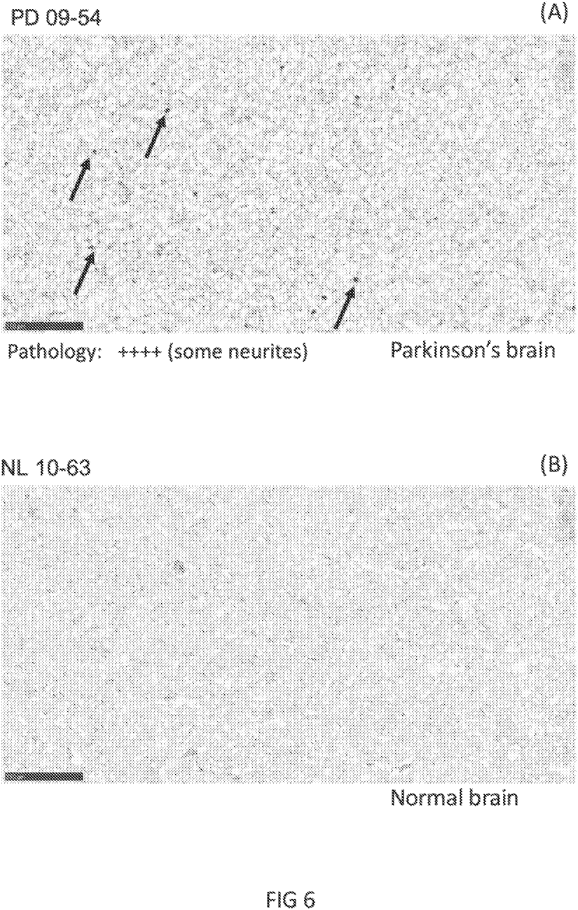

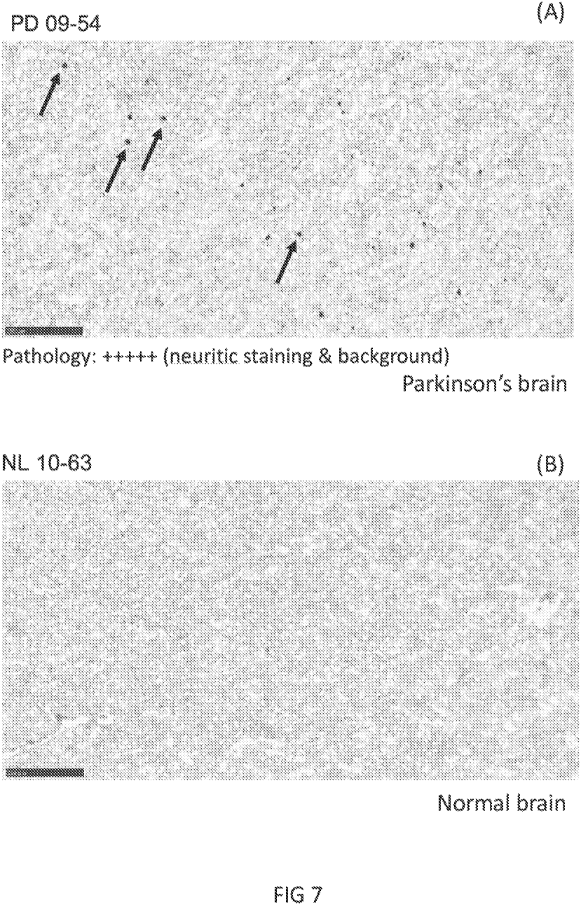

Results demonstrate alpha-synuclein pathologies were identified based on their typical histopathological characteristics. Such pathologies were absent from tissues incubated with control mouse serum. Also, normal, non-diseased tissue had no such pathological staining after incubation with the sera from vaccinated mice. FIG. 5(A) shows staining of synuclein pathology in fresh frozen human Parkinson’s brain tissue versus normal tissue FIG. 6(B) using a 1:300 dilution of serum from mice vaccinated with SEQ ID NO:78. FIG. 6(A) shows staining of synuclein pathology in fresh frozen human Parkinson’s brain tissue versus normal tissue FIG. 6(B) using a 1:300 dilution of serum from mice vaccinated with SEQ ID NO:79. FIG. 7(A) shows staining of synuclein pathology in fresh frozen human Parkinson’s brain tissue versus normal tissue FIG. 7(B), using a 1:300 dilution of serum from mice vaccinated with SEQ ID NO:81. Table 6 summarize the results of synuclein pathology staining using mouse sera from animals vaccinated with the specified peptides.

TABLE 6

Immunohistochemistry staining and titer results for mouse serum samples

| Animal | SEQ ID NO | Construct | Titer* | IHC Staining (1/300) |
|---|---|---|---|---|
| 13-1 | 76 | PDNEAYEGGC | 10000 | ++ |
| 13-2 | 76 | PDNEAYEGGC | 27000 | ++++ |
| 14-1 | 77 | DPDNEAYEGGC | 3000 | +/- |
| 14-4 | 77 | DPDNEAYEGGC | 22000 | +++ |
| 1-2 | 78 | PDNEAYERRDPDNEAYGGC | 200000 | ++++ |
| 1-3 | 78 | PDNEAYERRDPDNEAYGGC | 150000 | ++ |
| 1-4 | 78 | PDNEAYERRDPDNEAYGGC | 125000 | ++++ |
| 2-2 | 79 | DPDNEAYRRPDNEAYEGGC | 200000 | ++++ |
| 2-3 | 79 | DPDNEAYRRPDNEAYEGGC | 125000 | ++++ |
| 3-1 | 80 | PDNEAYERRTGFVKKDGGC | 250000 | - |
| 3-2 | 80 | PDNEAYERRTGFVKKDGGC | 300000 | - |
| 3-3 | 80 | PDNEAYERRTGFVKKDGGC | 225000 | +/- |
| 3-4 | 80 | PDNEAYERRTGFVKKDGGC | 175000 | - |
| 4-1 | 81 | TGFVKKDRRDPDNEAYEGGC | 225000 | ++++ |
| 4-3 | 81 | TGFVKKDRRDPDNEAYEGGC | 75000 | +++ |
| 4-4 | 81 | TGFVKKDRRDPDNEAYEGGC | 50000 | +++ |

- : negative + : weak ++ : moderate +++ : moderately strong ++++ : strong +++++: very strong

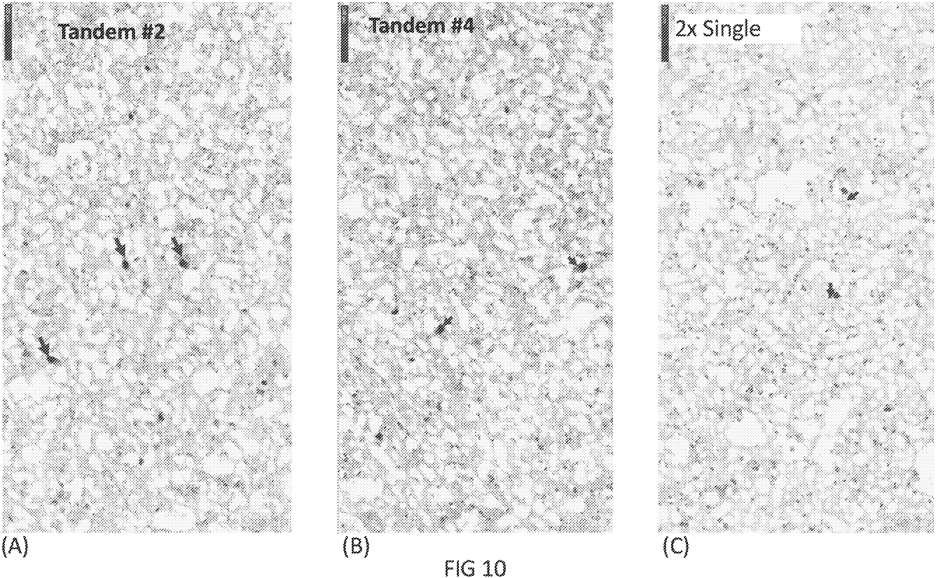

Experiments were repeated for SEQ ID NO:77, SEQ ID NO:79 and SEQ ID NO:81. FIG. 10(A), FIG. 10(B) and FIG. 10(C) show staining of synuclein pathology in fresh frozen human Parkinson’s brain tissue for SEQ ID NO:79, SEQ ID NO:81 and SEQ ID NO:77, respectively, including 2x dosing for monomeric alpha-synuclein peptide (SEQ ID NO:77) to compensate for having a single alpha-synuclein peptide compared to two for the tandem sequences. All samples bound to (stained) alpha-synuclein pathology, with polypeptides of SEQ ID NO:79 and SEQ ID NO:81 staining more efficiently in this experiment. Table 7 summarize the results of synuclein pathology staining using mouse sera from animals vaccinated with the specified peptides.

TABLE 7

Immunohistochemistry staining results for mouse serum samples

| Sample | SEQ ID NO | Mouse | Alpha-Syn Pathology -Cell Bodies | Alpha-Syn Pathology -Neurites |
|---|---|---|---|---|
| “Tandem 2” | 79 | 1 | +++++ ++ | ++ |
| “Tandem 2” | 79 | 2 | - | - |
| “Tandem 2” | 79 | 3 | +++ | +++ |
| “Tandem 2” | 79 | 4 | ++ | ++ |
| “Tandem 2” | 79 | 5 | ++++ | ++++ |
| “Tandem 2” | 79 | 6 | - | ++++ |
| “Tandem 2” | 79 | 7 | ++ | ++ |
| “Tandem 4” | 81 | 8 | +++ | ++ |
| “Tandem 4” | 81 | 9 | ++++ | ++++ |
| “Tandem 4” | 81 | 10 | ++++ | +++ |
| “Tandem 4” | 81 | 11 | +++ | + |
| “Tandem 4” | 81 | 12 | ++++ | +++ |
| “Tandem 4” | 81 | 13 | +++ | + |
| “Single 2x dose” | 77 | 14 | ++ | - |
| “Single 2x dose” | 77 | 15 | ++ | + |
| “Single 2x dose” | 77 | 16 | +++ | + |
| “Single 2x dose” | 77 | 17 | +++ | + |
| “Single 2x dose” | 77 | 18 | ++ | - |
| “Single 2x dose” | 77 | 19 | ++ | - |

- : negative + : weak ++ : moderate +++ : moderately strong ++++ : strong +++++ : very strong

### Example 4: Synuclein Preparation

### Manufacture of Synuclein

The bacterial expression vector, pET21d, containing the open reading frame (ORF) for human alpha-synuclein (GenBank ID: BC108275.1), was used for expression and identity were confirmed by sequencing. For expression and purification of recombinant alpha-synuclein, expression in BL21 E. coli cells was induced with 0.5 mM IPTG, and cells were lysed by sonication or microfluidizer. Lysates were cleared by boiling to generate a lysate enriched in heat stable alpha-synuclein, and this lysate was resolved by anion exchange chromatography. Pooled alpha-synuclein-pure fractions were dialyzed into PBS for storage.

### Aggregation of Synuclein

Aggregation of monomeric synuclein was induced by addition of 2.5% (v/v) HFIP (Sigma) to 2.6 mg/mL purified synuclein in PBS. Alpha-synuclein proteins with HFIP were incubated in a 37° C. thermomixer set to 1000 rpm for 4 days to induce aggregation. Before further use, the HFIP was allowed to evaporate off in a sample in hood, and the amyloid content was checked using a thioflavin T (ThT) assay.

### pHrodo Labeling of Synuclein

Aggregated and monomeric alpha-synuclein samples were labeled using pHrodo red (Invitrogen) for 1 hour at room temperature in the dark. Free dye was removed via extensive dialysis for the monomer sample and via ultracentrifugation of the aggregated synuclein and repeated rinsing of the resultant pellet. The aggregated alpha-synuclein was resuspended in PBS in the original volume and bath sonicated (QSonica) at 10% power for intervals of 10 seconds on and 5 seconds rest for 3 minutes of sonication time. The degree of labeling was quantified according to manufacturer’s instructions and determined to be 2 dye: 1 protein for both monomer and aggregated alpha synuclein.

### Example 5: B103 Blocking Assay

Ability of oligoclonal and polyclonal serum to block the binding of synuclein to B103 neuronal cell line was assessed.

Before use, purified pHrodo-labeled alpha-synuclein was resuspended in growth media to achieve a 10 ug/mL, a 4x working concentration, prior to binding to IgG cuts.

IgG cuts were diluted in growth media (4x working concentration of 1,200 ug/ml; n=3 per group). The 10 ug/ml pHrodo-labeled alpha-synuclein aggregates were added 1:1 (25 µl/sample) to IgG cuts (25 µl/sample). Antibody/alpha-synuclein mixtures were incubated at room temperature for 30 min.

B103 neuroblastoma cells were washed with warm sterile PBS and lifted with Versene/ETDA incubation for 5 min at 37° C., 5% CO₂. Cells were centrifuged at 200x g for 5 min and resuspended to 1,000,000 cells/ml in growth media (50 µl/sample). 50 µl of B103 cells were added to 50 µl antibody/alpha-synuclein mixtures in 96 well v-bottom plates. Following a 3 hour incubation at 37° C. at 5% CO₂, cells were washed three times with growth media (200x g; 5 min spins), incubated for 10 min at 37° C. at 5% CO₂, washed twice in FACS buffer (1% FBS in PBS; no Mg or Ca), and resuspended in 100 µl FACS buffer for flow cytometric analysis of labeled alpha-synuclein uptake by B103 cells.

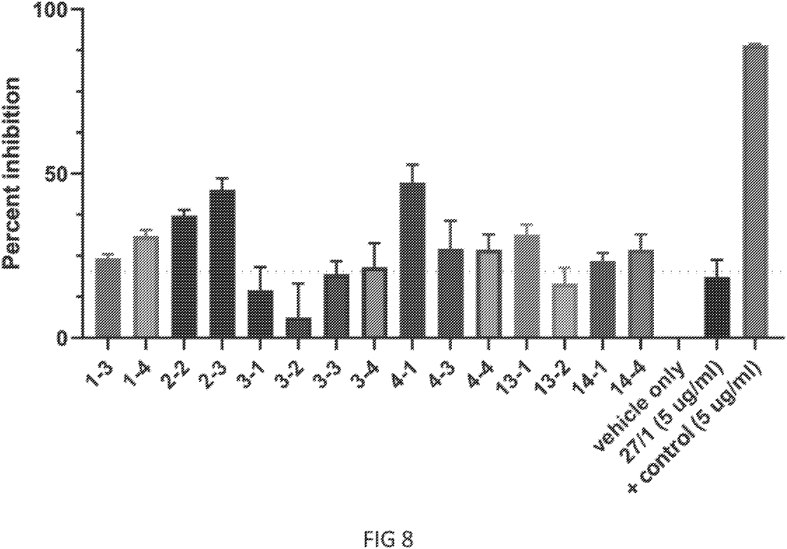

FIG. 8 shows that tandem constructs 2 (SEQ ID NO:79) and 4 (SEQ ID NO:81) were most efficient at inhibiting uptake of alpha-synuclein by B103 cells.

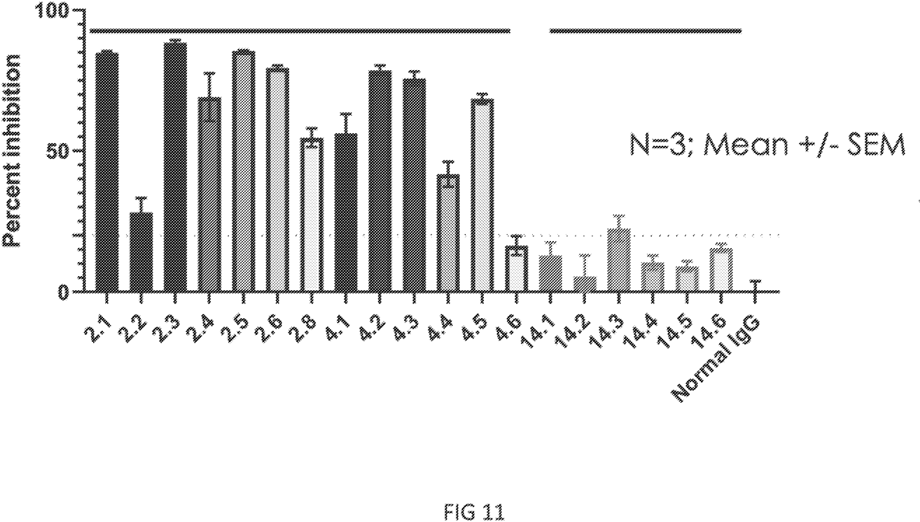

FIG. 11 again shows that tandem constructs 2 (SEQ ID NO:79) and 4 (SEQ ID NO:81) were most efficient at inhibiting uptake of alpha-synuclein by B103 cells. Single alpha-synuclein construct 14 (SEQ ID NO:77) did not appreciably inhibit synuclein binding in this experiment.

### Example 6: Flow Cytometry Analysis

The uptake of alpha-synuclein aggregates by B103 rat neuroblastoma cells was evaluated by flow cytometry for pHrodo-labeled fluorescently activated alpha-synuclein. Alpha-synuclein internalization was measured as mean fluorescence intensity (MFI; normalized to 100%). Normalization of MFI to 100% involved subtraction of background cellular MFI signal (no pHrodo Tau added) and setting the highest MFI signal to 100% (isotype control).

Although various specific embodiments of the present invention have been described herein, it is to be understood that the invention is not limited to those precise embodiments and that various changes or modifications can be affected therein by one skilled in the art without departing from the scope and spirit of the invention.

In each of the embodiments of the peptide described herein, the peptide may comprise, consist, or consist essentially of the recited sequences. Thus, incorporated in this disclosure (see Table 8) are the following sequences that can be part of the compositions comprising an alpha-synuclein (poly)peptide as disclosed herein.

TABLE 8

SEQUENCES
SEQ ID NO:01
Alpha-synuclein isoform NACP140 [Homo sapiens]
NCBI Reference Sequence: NP_000336.1
MDVFMKGLSK AKEGVVAAAE KTKQGVAEAA GKTKEGVLYV GSKTKEGVVH GVATVAEKTK
EQVTNVGGAV VTGVTAVAQK TVEGAGSIAA ATGFVKKDQL GKNEEGAPQE GILEDMPVDP
DNEAYEMPSE EGYQDYEPEA
C-Terminal Region:

| VDPDNEAYEM | (SEQ ID NO:02), |
|---|---|
| VDPDNEAYE | (SEQ ID NO:03), |
| VDPDNEAY | (SEQ ID NO:04), |
| VDPDNEA | (SEQ ID NO:05), |
| VDPDNE | (SEQ ID NO:06), |
| VDPDN | (SEQ ID NO:07), |
| VDPD | (SEQ ID NO:08), |
| VDP | (SEQ ID NO:09), |
| DPDNEAYEM | (SEQ ID NO:10), |
| DPDNEAYE | (SEQ ID NO:11), |
| DPDNEAY | (SEQ ID NO:12), |
| DPDNEA | (SEQ ID NO:13), |
| DPDNE | (SEQ ID NO:14), |
| DPDN | (SEQ ID NO:15), |
| DPD | (SEQ ID NO:16), |
| PDNEAYEM | (SEQ ID NO:17), |
| PDNEAYE | (SEQ ID NO:18), |
| PDNEAY | (SEQ ID NO:19), |
| PDNEA | (SEQ ID NO:20), |
| PDNE | (SEQ ID NO:21), |
| PDN | (SEQ ID NO:22), |
| DNEAYEM | (SEQ ID NO:23), |
| DNEAYE | (SEQ ID NO:24), |
| DNEAY | (SEQ ID NO:25), |
| DNEA | (SEQ ID NO:26), |
| DNE | (SEQ ID NO:27), |
| NEAYEM | (SEQ ID NO:28), |
| NEAYE | (SEQ ID NO:29), |
| NEAY | (SEQ ID NO:30), |
| NEA | (SEQ ID NO:31), |
| EAYEM | (SEQ ID NO:32), |
| EAYE | (SEQ ID NO:33), |
| EAY | (SEQ ID NO:34), |
| AYEM | (SEQ ID NO:35), |
| AYE | (SEQ ID NO:36), |
| YEM | (SEQ ID NO:37), |
| NAC-Region: | |
| ATGFVKKDQL | (SEQ ID NO:38), |
| ATGFVKKDQ | (SEQ ID NO:39, |
| ATGFVKKD | (SEQ ID NO:40), |
| ATGFVKK | (SEQ ID NO:41), |
| ATGFVK | (SEQ ID NO:42), |
| ATGFV | (SEQ ID NO:43), |
| ATGF | (SEQ ID NO:44), |
| ATG | (SEQ ID NO:45), |
| TGFVKKDQL | (SEQ ID NO:46), |
| TGFVKKDQ | (SEQ ID NO:47), |
| TGFVKKD | (SEQ ID NO:48), |
| TGFVKK | (SEQ ID NO:49), |
| TGFVK | (SEQ ID NO:50), |
| TGFV | (SEQ ID NO:51), |
| TGF | (SEQ ID NO:52), |
| GFVKKDQL | (SEQ ID NO:53), |
| GFVKKDQ | (SEQ ID NO:54), |
| GFVKKD | (SEQ ID NO:55), |
| GFVKK | (SEQ ID NO:56), |
| GFVK | (SEQ ID NO:57), |
| GFV | (SEQ ID NO:58), |
| FVKKDQL | (SEQ ID NO:59), |
| FVKKDQ | (SEQ ID NO:60), |
| FVKKD | (SEQ ID NO:61), |
| FVKK | (SEQ ID NO:62), |
| FVK | (SEQ ID NO:63), |
| VKKDQL | (SEQ ID NO:64), |
| VKKDQ | (SEQ ID NO:65), |
| VKKD | (SEQ ID NO:66), |
| VKK | (SEQ ID NO:67), |
| KKDQL | (SEQ ID NO:68), |
| KKDQ | (SEQ ID NO:69), |
| KKD | (SEQ ID NO:70), |
| KDQL | (SEQ ID NO:71), |
| KDQ | (SEQ ID NO:72), |
| CPDNEAYE | (SEQ ID NO:73), |
| DPDNEAYC | (SEQ ID NO:74), |
| CGFVKKDQ | (SEQ ID NO:75), |
| PDNEAYEGGC | (SEQ ID NO:76), |
| DPDNEAYEGGC | (SEQ ID NO:77), |
| PDNEAYERRDPDNEAYGGC | (SEQ ID NO:78), |
| DPDNEAYRRPDNEAYEGGC | (SEQ ID NO:79), |
| PDNEAYERRTGFVKKDGGC | (SEQ ID NO:80), |
| TGFVKKDRRDPDNEAYEGGC | (SEQ ID NO:81), |
| Arg-Val-Arg-Arg | (RVRR; SEQ ID NO:82), |
| Gly-Ala-Gly-Ala | (GAGA; SEQ ID NO:83), |
| Ala-Gly-Ala-Gly | (AGAG; SEQ ID NO:84), |
| Lys-Gly-Lys-Gly | (KGKG; SEQ ID NO:85), |
| Gly-Gly-Gly-Ser | (GGGS; SEQ ID NO:86), and |
| Gly-Gly-Gly-Gly-Ser | (GGGGS; SEQ ID NO:87) . |

What is claimed is:

1. A peptide comprising 3-10 amino acids from residues 81-140 of SEQ ID NO:01 and optionally a C-terminal cysteine.

2. The peptide of claim 1, wherein the peptide is from residues 111-131 of SEQ ID NO:01 and optionally a C-terminal cysteine.

3. The peptide of claim 2, wherein the peptide comprises an amino acid sequence selected from the group consisting any one of SEQ ID NO:02 through SEQ ID NO:37.

4. The peptide of claim 1, wherein the peptide is from residues 83-106 of SEQ ID NO:01 and optionally a C-terminal cysteine.

5. The peptide of claim 4, wherein the peptide comprises an amino acid sequence selected from the group consisting of any one of SEQ ID NO:38 through SEQ ID NO:72.

6. The peptide of claim 1, wherein the peptide comprises an amino acid sequence selected from the group consisting of any one of SEQ ID NO:02 through SEQ ID NO:72.

7. The peptide of claim 1, comprising an amino acid sequence of any one of DPDNEAYE (SEQ ID NO:48), DPDNEAY (SEQ ID NO:49), PDNEAYE (SEQ ID NO:55), ATGFVKK (SEQ ID NO:41), TGFVKKD (SEQ ID NO:48), GFVKKDQ (SEQ ID NO:54) or DPDNEAYC (SEQ ID NO:74).

8. The peptide of any one of claims 1 to 7, optionally comprising an N-terminal cysteine.

9. The peptide of claim 8, comprising an amino acid sequence of CPDNEAYE (SEQ ID NO:73) or CGFVKKDQ (SEQ ID NO:75).

10. A peptide comprising an amino acid sequence of PDNEAYEGGC (SEQ ID NO:76), DPDNEAYEGGC (SEQ ID NO:77), PDNEAYERRDPDNEAYGGC (SEQ ID NO:78), DPDNEAYRRPDNEAYEGGC (SEQ ID NO:79), PDNEAYERRTGFVKKDGGC (SEQ ID NO:80), or TGFVKKDRRDPDNEAYEGGC (SEQ ID NO:81).

11. The peptide of any one of claims 1 to 10, further comprising a linker at a C-terminal portion of the peptide.

12. The polypeptide of claim 11, wherein the linker comprises an amino acid sequence.

13. The peptide of claim 12, wherein the linker comprises an amino acid sequence selected from the group consisting of GG, GGG, AA, AAA, KK, KKK, SS, SSS, AGAG (SEQ ID NO:84), GG, GAGA (SEQ ID NO:83) and KGKG (SEQ ID NO:85).

14. The peptide of any one of claims 1 to 13, wherein the polypeptide or linker to the carrier, if present, further comprises a C-terminal cysteine (C).

15. The peptide of any one of claims 1 to 14, wherein the peptide further comprises a blocked amine at the N-terminus.

16. The polypeptide of any one of claims 1 to 15, wherein the second peptide comprises 5-10 amino acids.

17. An immunotherapy composition, comprising one or more of the peptides of any one of claims 1 to 16.

18. The immunotherapy composition of claim 17, wherein the one or more peptides further comprises a linker to a carrier at a C-terminal portion of the peptide.

19. The immunotherapy composition of claim 18, wherein the linker comprises an amino acid sequence selected from the group consisting of GG, GGG, AA, AAA, KK, KKK, SS, SSS, AGAG (SEQ ID NO:84), GG, GAGA (SEQ ID NO:83) and KGKG (SEQ ID NO:85).

20. The immunotherapy composition of either of claim 18 or 19, wherein the carrier comprises serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

21. The immunotherapy composition of claim 20, wherein the carrier is CRM197.

22. The immunotherapy composition of claim 20, wherein the carrier is diphtheria toxoid.

23. The immunotherapy composition of any one of claims 17 to 22, further comprising at least one pharmaceutically acceptable diluent.

24. The immunotherapy composition of any one of claims 17 to 23, further comprising a multiple antigen presenting system (MAP).

25. The immunotherapy composition of claim 24, wherein the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

26. A pharmaceutical composition comprising (a) one or more of the polypeptide of any one of claims 1 to 16 or (b) the immunotherapy composition of any of claims 17 to 25 and at least one adjuvant.

27. The pharmaceutical composition of claim 26, wherein the adjuvant is selected from the group consisting of aluminum hydroxide, aluminum phosphate, aluminum sulfate, 3 De-O-acylated monophosphoryl lipid A (MPL), QS-21, TQL1055, QS-18, QS-17, QS-7, Complete Freund’s Adjuvant (CFA), Incomplete Freund’s Adjuvant (IFA), oil in water emulsions (such as squalene or peanut oil), CpG, polyglutamic acid, polylysine, AddaVax™, MF59®, and combinations thereof.

28. The pharmaceutical composition of claim 27, wherein the adjuvant is QS-21 or TQL1055.

29. The pharmaceutical composition of claim 27, wherein the adjuvant is MPL.

30. The pharmaceutical composition of claim 27, wherein the adjuvant is a combination of MPL and QS-21 or a combination of MPL and TQL1055.

31. The pharmaceutical composition of any one of claims 26 to 30, wherein the adjuvant comprises a liposomal formulation.

32. The pharmaceutical composition of any one of claims 26 to 31, wherein the composition comprises at least one pharmaceutically acceptable diluent.

33. The pharmaceutical composition of any one of claims 26 to 32, comprising a multiple antigen presenting system (MAP).

34. The pharmaceutical composition of claim 33, wherein the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

35. A nucleic acid comprising a nucleic acid sequence encoding a polypeptide of any one of claims 1 to 16 or the immunotherapy composition of any one of claims 17 to 25.

36. A nucleic acid immunotherapy composition comprising the nucleic acid of claim 35 and at least one adjuvant.

37. A method of treating or effecting prophylaxis of an alpha-synucleinopathy in a subject, comprising administrating to the subject the immunotherapy composition of any one of claims 17 to 25 or the pharmaceutical compositions of any one of claims 26 to 34.

38. A method of blocking the uptake of alpha-synuclein by neurons, inhibiting cell-to-cell transmission of alpha-synuclein seeds or inhibiting or reducing aggregation of alpha-synuclein in a subject having or at risk of developing an alpha-synucleinopathy, comprising, administering to the subject the immunotherapy composition of any one of claims 17 to 25 or the pharmaceutical composition of any one of claims 26 to 34.

39. A method of treating or effecting prophylaxis of an alpha-synucleinopathy in a subject, comprising administrating to the subject the nucleic acid immunotherapy composition of claim 36.

40. A method of blocking the uptake of alpha-synuclein by neurons, inhibiting cell-to-cell transmission of alpha-synuclein seeds or inhibiting or reducing aggregation of alpha-synuclein in a subject having or at risk of developing an alpha-synucleinopathy, comprising administering to the subject the nucleic acid immunotherapy composition of claim 36.

41. The method of any one of claims 37 to 40, wherein the alpha-synucleinopathy is selected from the group consisting of Parkinson’s disease, dementia with Lewy bodies (DLB), multiple system atrophy (MSA), Alzheimer’s disease, and neurodegeneration with brain iron accumulation (NBIA).

42. The method of claim 41, wherein the multiple system atrophy (MSA) is selected from the group consisting of the MSA-parkinsonian (MSA-P) variant and the MSA-cerebellar (MSA-C) variant.

43. The method of claim 41, wherein the Parkinson’s disease is Parkinson’s disease with dementia (PDD).

44. The method of claim 41, wherein the Alzheimer’s disease is the Lewy body variant of Alzheimer’s disease.

45. The method of any of claims 37 to 44, further comprising repeating the administering at least a second time, at least a third time, at least a fourth time, at least a fifth time, or at least a sixth time.

46. The method of claim 45, further comprising repeating the administering at an interval of about 14 days, or about 21 to about 28 days, or about quarterly, or about biannually, or about annually.

47. A method of inducing an immune response in an animal, comprising administering to the animal any one of the polypeptide of claims 1 to 16, the immunotherapy composition of claims 17 to 25, the pharmaceutical compositions of claims 26 to 34 or the nucleic acid immunotherapy composition of claim 36 in a regimen effective to generate an immune response comprising antibodies that specifically bind to alpha-synuclein.

48. The method of claim 47, wherein the immune response comprises antibodies that specifically bind to alpha-synuclein.

49. The method of any of claims 47 to 48, wherein the inducing the immune response comprises antibodies that specifically bind to the C-terminal region of alpha-synuclein.

50. The method of any of claims 47 to 48, wherein the inducing the immune response comprises antibodies that specifically bind to the NAC region of alpha-synuclein.

51. An immunization kit comprising the immunotherapy composition of any of claims 17 to 25.

52. The kit of claim 51, further comprising an adjuvant.

53. The kit of claim 52, wherein the immunotherapy composition is in a first container and the adjuvant is in a second container.

54. A kit comprising the nucleic acid immunotherapy composition of claim 36.

55. The kit of claim 54, further comprising an adjuvant.

56. The kit of claim 55, wherein the nucleic acid is in a first container and the adjuvant is in a second container.
